(19) United States
(12) Patent Application Publication
(10) Pub. No.: US 2009/0155325 A1
(43) Pub. Date: Jun. 18, 2009

(54) FORMULATION AND PRODUCTS FOR PROMOTING SKIN CLEANLINESS AND HEALTH

(75) Inventors: Scott W. Wenzel, Neenah, WI (US); David William Koenig, Menasha, WI (US); Douglas R. Hoffman, Greenville, WI (US); Candace Dyan Krautkramer, Neenah, WI (US); Brian Thomas, Appleton, WI (US); Cheryl Mocadlo, New London, WI (US); Chelsea Magin, Gainesville, FL (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: Kimberly-Clark Worldwide, Inc., Neenah, WI (US)

(21) Appl. No.: 11/957,169

(22) Filed: Dec. 14, 2007

(51) Int. Cl. A61F 13/15; A61K 47/02; A61K 47/26; A61P 17/00; A61K 47/06

(52) U.S. Cl. 424/402; 514/772; 514/769; 514/781; 514/782

(57) ABSTRACT

The present disclosure generally relates to a formulation. More particularly, the formulation may be applied to skin and/or used in combination with a personal care product to improve absorbance and leakage control of the personal care product, and to promote a more positive feel and comfort through improved skin cleanliness and health.

### BACKGROUND OF THE DISCLOSURE

This disclosure relates generally to a formulation which may be applied to the skin of a user. More particularly, the formulation promotes a more positive feel and comfort through improved skin cleanliness and health. The formulation may be applied directly to the skin or incorporated into personal care products such as wipes or absorbent articles.

A large variety of personal care absorbent articles or products are known. Examples of such products include feminine care articles, diapers, children's training pants, adult incontinence articles, and the like. The major function of absorbent articles is to absorb and contain body exudates. Such articles are thus intended to prevent body exudates from soiling, wetting, or otherwise contaminating clothing or other articles, such as bedding, that come in contact with the wearer.

The most common mode of failure for such products is the leakage of fluids when using the articles, particularly from around the side edges of the article, or out of the gaps between the article and the wearer's leg or waist, in the case of diapers or training pants. Leakage can be caused by exudates never coming in contact with or remaining on the top layers of absorbent material. For instance, exudates such as urine, menstrual fluids, etc. may wick along the body and remain on the skin and/or hair of the article's user, thus preventing the exudates from being taken up by the absorbent article, and may ultimately result in leakage of the absorbent article. Furthermore, exudates that become attached to the skin and/or hair can result in an uncomfortable, unclean feeling of the user's skin.

In addition to soiling resulting from exudates, a variety of microflora may also be found on the surface of skin at any given time. The adherence to the skin of problem microflora, such as pathogenic bacteria and yeast has been associated with numerous ailments, including skin infections, diaper rash, urinary or vaginal infections, and malodors.

Various products are commercially available to clean the surface of skin and to remove exudates and problem microflora there from. For example, wet wipes are commonly used to remove urine, menses, mucous, vaginal discharges, complex liquids, and microflora from the skin of humans. These wet wipes typically comprise a surfactant system for cleaning the skin and removing liquids and solids located thereon. In some cases, wet wipes may comprise an antibacterial agent, such as an organic acid, which can be used in combination with the surfactant to kill bacteria located on the skin's surface. Also, various antibacterial soaps and cleansers are available to cleanse hands and kill microflora adhered to the skin's surface. These antibacterial soaps are generally highly effective in killing bacteria located on the skin.

Although various conventional formulations are available to clean and sanitize skin, such formulations can sometimes be harsh on the skin, particularly after repeated use. In some cases, skin can become dry or chaffed, and the use of the formulation and associated product must be discontinued until the skin heals.

There has thus been a continued need for a formulation which can be applied to the skin of a user that can help keep problem microflora and undesired materials such as exudates from adhering and remaining on the skin, while also promoting a more positive feel and comfort to the user through improved skin cleanliness and skin health.

### SUMMARY OF THE DISCLOSURE

The present disclosure relates generally to a formulation which may be applied to the skin of a user. More particularly, the formulation promotes a more positive feel and comfort through improved skin cleanliness and health. The formulation may be applied directly to the skin or incorporated into personal care products such as wipes or absorbent articles.

In one aspect, the present disclosure is directed to a formulation for promoting skin cleanliness and health. The formulation comprises from about 0.01% (by total weight of the formulation) to about 10% (by total weight of the formulation) of a coefficient of friction modulator; from about 1% (by total weight of the formulation) to about 15% (by total weight of the formulation) of a moisturizer; from about 0.01% (by total weight of the formulation) to about 10% (by total weight of the formulation) of an elasticity modulator; from about 0.01% (by total weight of the formulation) to about 10% (by total weight of the formulation) of a skin texture modulator; and from about 1% (by total weight of the formulation) to about 99% (by total weight of the formulation) of a pharmaceutically acceptable carrier.

In another aspect, the present disclosure is directed to a wet wipe for promoting skin cleanliness and health. The wet wipe comprises a wipe substrate; and a liquid formulation. The liquid formulation comprises from about 0.01% (by total weight of the formulation) to about 10% (by total weight of the formulation) of a coefficient of friction modulator; from about 1% (by total weight of the formulation) to about 15% (by total weight of the formulation) of a moisturizer; from about 0.01% (by total weight of the formulation) to about 10% (by total weight of the formulation) of an elasticity modulator; from about 0.01% (by total weight of the formulation) to about 10% (by total weight of the formulation) of a skin texture modulator; and from about 1% (by total weight of the formulation) to about 99% (by total weight of the formulation) of a pharmaceutically acceptable carrier.

In another aspect, the present disclosure is directed to an absorbent article. The absorbent article comprises an absorbent substrate; and a formulation comprising from about 0.01% (by total weight of the formulation) to about 10% (by total weight of the formulation) of a coefficient of friction modulator; from about 1% (by total weight of the formulation) to about 15% (by total weight of the formulation) of a moisturizer; from about 0.01% (by total weight of the formulation) to about 10% (by total weight of the formulation) of an elasticity modulator; from about 0.01% (by total weight of the formulation) to about 10% (by total weight of the formulation) of a skin texture modulator; and from about 1% (by total weight of the formulation) to about 99% (by total weight of the formulation) of a pharmaceutically acceptable carrier.

In still another aspect, the present disclosure is directed to a system for promoting skin cleanliness and health. The system may comprise a formulation, a wipe, an absorbent article, or some combination thereof, as described herein.

Other objects and features will be in part apparent and in part pointed out hereinafter.

### BRIEF DESCRIPTION OF THE FIGURES

FIG. 1 shows a representative, partially cut-away, top, plan view of a body-side of an absorbent article in which separately side-panels or wings are assembled to the article and arranged in a storage position.

FIG. 1A shows a representative, bottom, plan view of a garment-side of the absorbent article illustrated in FIG. 1.

FIG. 1B shows an expanded, schematic view of a representative, transverse cross-section of the absorbent article illustrated in FIG. 1.

FIG. 1C shows an expanded, schematic view of a representative, longitudinal cross-section of the absorbent article illustrated in FIG. 1.

FIG. 2 shows a representative, partially cut-away, top, plan view of a body-side of an absorbent article having side-panels or wings that have been unitarily formed with one or more components of the article, where the wings include a system of interengaging mechanical fasteners.

FIG. 2A shows a representative, bottom, plan view of a garment-side of the absorbent article illustrated in FIG. 2.

FIG. 2B shows an expanded, schematic view of a representative, transverse cross-section of the absorbent article illustrated in FIG. 2.

FIG. 2C shows an expanded, schematic view of a representative, longitudinal cross-section of the absorbent article illustrated in FIG. 2.

FIG. 3 shows a representative, partially cut-away, top, plan view of a bodyside of an absorbent article having side-panels or wings that have been unitarily formed with one or more components of the article, where the wings include a system of adhesive fasteners.

FIG. 3A shows a representative, bottom, plan view of a garment-side of the absorbent article illustrated in FIG. 3.

FIG. 3B shows an expanded, schematic view of a representative, transverse cross-section of the absorbent article illustrated in FIG. 3.

FIG. 3C shows an expanded, schematic view of a representative, longitudinal cross-section of the absorbent article illustrated in FIG. 3.

FIG. 4 shows a representative, top view of a bodyside of an absorbent article having a selected pattern of embossments formed into the article.

FIG. 4A shows a representative, top view of a bodyside of an absorbent article having another distribution of embossments formed into the article.

FIG. 4B shows an expanded, schematic view of a representative, transverse cross-section of the absorbent article illustrated in FIG. 4.

FIG. 5 shows a representative, top view of a bodyside of an absorbent article having a selected pattern of apertures formed into the bodyside surface of the article.

FIG. 5A shows a representative, top view of a bodyside of an absorbent article having another distribution of apertures formed into the bodyside surface of the article.

FIG. 5B shows a schematic, expanded view of a representative, transverse cross-section of the absorbent article illustrated in FIG. 5.

FIGS. 6-8 are graphs showing the force curve (frictional force (gf) vs. time) for silk (FIG. 6), spunbond nonwoven (FIG. 7), and synthetic skin substrate (FIG. 8) for the coefficient of friction tests performed as described in Example 3.

FIGS. 9-10 are charts showing the difference in conductance (FIG. 9) and skin roughness (FIG. 10) measurements for skin treated with formulations of the present disclosure, as compared to untreated skin, as described in Example 4.

### DETAILED DESCRIPTION OF THE DISCLOSURE

The present disclosure generally relates to a formulation. More particularly, the formulation may be applied to skin and/or used in combination with a personal care product to improve absorbency and leakage control of the personal care product, and to promote a more positive feel and comfort through improved skin cleanliness and health.

In accordance with the present disclosure, it has been discovered that a formulation comprising a coefficient of friction (COF) modulator, moisturizer, elasticity modulator, skin texture modulator, and optionally an anti-adherence agent and/or viscoelastic agent can improve skin cleanliness and health and promote a positive skin feel when applied to skin and/or to personal care products that come in contact with skin.

As noted above, body exudates such as menses, intermenstrual discharge, urine, fecal matter, and the like, may adhere to skin and hair and/or wick along the skin or hair, impairing the ability of absorbent articles to intercept and absorb the exudates. Advantageously, the formulations of the present disclosure can be used to reduce the adherence of body exudates to skin, mucosa, and hair, and to promote transfer of the exudate from the body into an absorbent article. More particularly, the formulation may be applied directly to the skin of a user, or may be used in combination with a personal care product such as a wipe or absorbent article. By using the formulation in combination with a wipe or absorbent article substrate, it is possible to transfer the formulation to the skin during use of the product and to reduce the adherence of exudates to the skin. By reducing exudate adherence, the degree of wicking along the body is reduced, thus improving the effectiveness of absorbent articles, and reducing absorbent article leakage. Furthermore, reducing the degree of exudate adherence improves the ease by which exudates may be removed from the skin.

Additionally, as noted above, microflora are often naturally found on the skin or mucosal surfaces of the body. In particular, problem microflora, such as pathogenic bacteria or yeast, are associated with numerous ailments, including skin infections, diaper rash, urinary or vaginal infections, and malodors, among others. It is thus often desirable to inhibit the adherence of such microflora to the surface of skin or mucosa. Problem microflora may include a variety of microorganisms, such as Gram negative bacteria, Gram positive bacteria, acid fast bacteria, Mycoplasma, fungi, yeast, and viruses. Some specific examples of problem microflora include Candida albicans, Proteus mirabilis, and Pseudomonas aeruginosa, among others. Another flora, Staphylococcus epidermidis, may become an opportunistic pathogen by spreading into the blood through breaks in skin barriers. It will be apparent to those skilled in the art that there are numerous other examples of microflora, other than those described herein, for which it may be desirable to inhibit the adherence, and that the adherence of these microflora may be controlled in a manner similar to that described herein. Advantageously, the formulations described herein not only reduce the degree of exudate adherence to skin, mucosa, and hair, but also may inhibit the adherence of problematic microflora to skin, thus improving skin health. The formulations and personal care products described herein can thus be used to reduce skin infections and treat compromised skin, as well as treat uncompromised skin, such that it will stay clean and hygienic.

In addition to reducing the degree of exudate and microflora adherence to skin, the formulations of the present disclosure also advantageously impart other skin health and comfort benefits. For instance, the formulations may help maintain or improve various skin properties, such as skin moisture, skin temperature, skin elasticity, and skin texture, as described more fully hereinafter.

Thus, in one aspect, the present disclosure is directed to a formulation comprising a coefficient of friction modulator, a moisturizer, an elasticity modulator, a skin texture modulator, and an anti-adherence agent. In addition, one or more of the composition components may alter the viscosity of the bodily exudates such that they are more efficiently transferred to the absorbent article and absorbed. The formulation may take a variety of forms including, without limitation, aqueous solutions, gels, balms, lotions, suspensions, creams, milks, salves, ointments, sprays, emulsions, oils, resins, foams, films, solid sticks, aerosols, and the like. In one aspect, the formulation may be applied directly to the skin.

In another aspect, the formulation of the present disclosure may be used in combination with a product, such as a personal care product. More particularly, the formulation may be incorporated into or onto a substrate, such as a wipe substrate, an absorbent substrate, a fabric or cloth substrate, or a tissue substrate, among others. For example, the formulation may be incorporated into personal care products, such as wipes, absorbent articles, bath tissues, cloths, and the like. In one preferred embodiment, the formulation is a liquid formulation that may be used in combination with a wipe substrate to form a wet wipe.

As noted above, the formulations of the present disclosure may advantageously comprise a coefficient of friction modulator. Oftentimes skin may become chaffed or otherwise damaged due to friction resulting from the skin rubbing against materials that come in contact with it, such as clothing, the top liner of an absorbent product (e.g., a diaper, feminine pad, and the like), or even other skin. It has now been discovered that the degree of gentleness of a material (e.g., an absorbent product, a wipe, skin, etc.) can be improved by treating the material with a formulation comprising a coefficient of friction modulator. Similar improvements in gentleness may be obtained by applying a formulation comprising a coefficient of friction modulator to skin contacting the material. In particular, the coefficient of friction modulator reduces irritation and skin damage that may otherwise result from the skin rubbing against the material. Advantageously, the coefficient of friction modulator also allows the formulations of the present disclosure to be applied to a user in a manner which is non-irritating to the skin.

Typically, the gentleness of a material can be determined by comparing the coefficient of friction, or the force required to start the material moving against skin, of the material to an appropriate control. The coefficient of friction value can be recorded as the force measurement that corresponds to the top of the initial peak of a force curve measured in grams force (gf). Lower coefficient of friction values indicate lower shear forces between the material and skin, and increased gentleness. Thus, as used herein, the term “coefficient of friction modulator” refers to components that lower both the static coefficient of friction (i.e., the amount of force required to start an object moving across a surface from a stationary position) and dynamic coefficient of friction (i.e., the amount of force required to keep an object moving across a surface).

For purposes of the present disclosure, the degree of gentleness of a material can be defined by a gentleness ratio. The gentleness ratio=static coefficient of friction of the material being tested/static coefficient of friction of a corduroy control. By expressing the degree of gentleness of a material in terms of the gentleness ratio, the gentleness of a material can be consistently measured and compared to measurements from other materials. Preferably, the materials treated with a formulation of the present disclosure will have a gentleness ratio of about 0.95 or less, and more preferably about 0.85 or less.

Examples of suitable coefficient of friction modulators include, for example, silicone compounds such as polydimethylsiloxane, dimethicone, silicone crosspolymers, silicone gums, dimethiconol, trimethylsilyl, amodimethicone, silsesquioxanes, alkylmethylsiloxanes, silicone wax, silanes, silica, and fumed silica; propylene glycol; 1,2-propanediol; stearalkonium chloride (i.e., stearyl dimethyl benzyl ammonium chloride); starch; cyclomethicone; mineral oil; petrolatum; polymethacrylate; polymethylmethacylate; aluminum starch octenylsuccinate, calcium starch octenylsuccinate, particulate blends such as DRYFLOW® ELITE LL (INCI: Aluminum Starch Octenylsuccinate (and) Lauroyl Lysine) (available from National Starch); DRYFLOW® ELITE BN (INCI: Aluminum Starch Octenylsuccinate (and) Boron Nitride) (available from National Starch); Nylon-12, Nylon-6; nanoparticles; cellulosics such as carboxymethylcellulose, ethyl cellulose, and hydroxyethylcellulose; silicone polymers; molybdenum disulfide; polytetrafluoroethylene; insoluble starch (available from National Starch); lipids; cross-linked alginate; dextrin; graphite; cross-linked vinylpyrrolidone homopolymers; chitin; polypropylene; pectin; talc; cationic polymers; and combinations thereof. These ingredients could be used in a variety of sizes and structures including, but not limited to micronized particles in spherical, amorphous, or other structures.

Preferably, the coefficient of friction modulator is selected from the group consisting of silicone compounds such as polydimethylsiloxane, dimethicone, silicone crosspolymers, silicone gums, dimethiconol, amodimethicone, silicone wax, cyclomethicone, particulates such as silsesquioxanes, and starch; polymethacrylate; polymethylmethacylate; Aluminum Starch Octenylsuccinate, Calcium Starch Octenylsuccinate, particulate blends such DRYFLOW® ELITE LL (INCI: Aluminum Starch Octenylsuccinate (and) Lauroyl Lysine) (available from National Starch); DRYFLOW® ELITE BN (INCI: Aluminum Starch Octenylsuccinate (and) Boron Nitride) (available from National Starch); Nylon-12; Nylon-6; talc; cationics such as stearalkonium chloride (i.e., stearyl dimethyl benzyl ammonium chloride); and glycols such as propylene glycol and butylene glycol; and combinations thereof. More preferably, the coefficient of friction modulator is dimethicone.

The coefficient of friction modulator may be present in the formulation in amounts of from about 0.01% (by total weight of the formulation) to about 10% (by total weight of the formulation), more preferably from about 0.1% (by total weight of the formulation) to about 8% (by total weight of the formulation), and still more preferably from about 1% (by total weight of the formulation) to about 6% (by total weight of the formulation).

In one particular embodiment, the coefficient of friction modulator is dimethicone and is present in the formulation in an amount of from about 0.01% (by total weight of the formulation) to about 10% (by total weight of the formulation), and more preferably in an amount of about 6% (by total weight of the formulation).

The formulations of the present disclosure may further comprise a moisturizer. It is generally undesirable for skin to have significantly more moisture or significantly less moisture (i.e., dry skin) than that which naturally occurs in healthy skin. Inclusion of a moisturizer in the formulations described herein will advantageously help maintain or improve appropriate skin moisture levels.

Preferably, the formulations will maintain or improve stratum corneum moisturization for at least about 4 hours, and more preferably for at least about 6 hours, and more preferably at least up to 24 hours after application of the formulation.

The degree of moisturization may be determined by measuring the electrical conductance of the skin, as set forth in the examples. Conductance is the cosmetic industry standard for measuring skin moisturization. In general, conductance is measured using electrodes that send a series of alternating electrical currents through the skin. Resistance to the currents indicates the water binding capacity of the stratum corneum (i.e., the moisture level), and provides a conductance reading. In general, a higher conductance reading indicates a higher level of moisture in the skin, and thus increased moisturization. It is generally preferable for the conductance readings of skin treated with a formulation described herein to be higher than baseline conductance readings of skin having no formulation applied thereto.

Examples of suitable moisturizers include, for example, glycerin, ethoxylated glycerin, sodium lactate, lactic acid, glycolic acid, urea, hydrolyzed proteins, hyaluronic acid, salicylic acid, phospholipids, propylene glycol, butylene glycol, caprylyl glycol, ethoxylated propylene glycol, glycerol, collagen, pyrrolidone carboxylic acid (PCA), sodium PCA, betaine, diglycerin, glucose, sucrose, xylitol, fructose, sorbitol, mannitol, hydrolyzed starch, and combinations thereof. Preferably, the moisturizer is selected from the group consisting of glycerin, lactic acid, sodium lactate, urea, hyaluronic acid, propylene glycol, glycerol, sodium PCA, betaine, sorbitol, sucrose, mannitol, and combinations thereof. In one particular embodiment, the moisturizer is glycerin.

The moisturizer may be present in the formulation in amounts of from about 1% (by total weight of the formulation) to about 15% (by total weight of the formulation), more preferably from about 2% (by total weight of the formulation) to about 10% (by total weight of the formulation), and still more preferably from about 3% (by total weight of the formulation) to about 9% (by total weight of the formulation), and more preferably is present in the composition in an amount of about 5% (by total weight of the formulation). In one preferred embodiment, the moisturizer is glycerin and is present in the formulation in an amount of about 5% (by total weight of the formulation).

The formulations described herein may further comprise an elasticity modulator. As used herein, the term “elasticity modulator” refers to ingredients that are added to the formulation to help maintain or improve the elasticity of the treated skin.

Methods for measuring skin elasticity are known in the art. One suitable method is set forth herein in the examples. In particular, elasticity may be expressed as the difference between the pressure required to pull the skin up 1 mm and the pressure required to pull the skin up 2.5 mm. In general, the lower the pressure difference, the more elastic the skin is. Elasticity may be measured using any suitable instrument such as a DermaLab Elasticity Probe (available from Cortex Technology, Hadsund, Denmark).

Examples of suitable elasticity modulators include, for example, β-glucan, glycerin, lactic acid, glycolic acid, urea, hydrolyzed proteins, dimethylaminoethanol, α-hydroxy acids, liposome collagen, collagen, Vitamin C, Vitamin E, Vitamin A and vitamin derivatives, elastin, peptides, and combinations thereof. Preferably, the elasticity modulator is selected from the group consisting of Vitamin C, Vitamin E, Vitamin A, vitamin derivatives, and combinations thereof. More preferably, the elasticity modulator is Vitamin E acetate (i.e., α-tocopheryl acetate).

The elasticity modulator may be present in the formulations in amounts of from about 0.01% (by total weight of the formulation) to about 10% (by total weight of the formulation), more preferably from about 0.1% (by total weight of the formulation) to about 8% (by total weight of the formulation), and more preferably from about 0.5% (by total weight of the formulation) to about 5% (by total weight of the formulation), and more preferably in an amount of about 1% (by total weight of the formulation). In one aspect, the elasticity modulator is Vitamin E acetate and is present in the formulation in an amount of from about 0.5% (by total weight of the formulation to about 5% (by total weight of the formulation, and more preferably is present in an amount of about 1% (by total weight of the formulation).

The formulation may further comprise a skin texture modulator. As used herein, the term “skin texture modulator is intended to refer to formulation components that impact the degree of skin smoothness. For instance, the skin texture modulator advantageously will decrease the roughness of skin treated with a formulation comprising the skin texture modulator as compared to untreated skin, resulting in smoother feeling skin. Preferably, the skin texture modulator will not cause a significant change in skin surface roughness as compared to normal, healthy skin.

Skin texture modulators affect the degree of skin smoothness by altering the skin topography. Specifically, skin texture modulators adhere to the skin surface and will deposit into crevices in the skin, leaving a smoother skin texture. Skin texture measurements (i.e., skin roughness) can be determined by using profilometry, as described in the examples. Preferably, the skin surface roughness measurement for skin treated with a formulation of the present disclosure will be lower than that for untreated “normal” skin (baseline), indicating a decrease in skin surface roughness.

Examples of suitable skin texture modulators include, for example, acrylics, acrylics multipolymer, cellulose nitrate, polypropylene (unmodified), polybutylene, ionomers, polyethylene (low density), polyethylene (medium density), Nylon-6, Nylon-12, styrene butadiene thermoplastic copolymer, polyvinylchloride (PVC) (rigid), polymethylmethacylate, aluminum starch octenylsuccinate, lauroyl lysine, calcium starch octenylsuccinate, boron nitride, polymethacrylate, nanoparticles, carboxymethylcellulose, micronized silicone polymers (available from Dow), graphite, cross-linked vinylpyrrolidone homopolymers, silica, ethylcellulose resins (available from Dow), micronized chitin, micronized polypropylene, pectin, hydroxyethylcellulose, talc, cationic polymers, silsesquioxanes, starch, polymethacrylate, cationics such as stearalkonium chloride (i.e., stearyl dimethyl benzyl ammonium chloride), glycols, and combinations thereof.

Preferably, the skin texture modulator is selected from the group consisting of polypropylene, polyethylene, Nylon-6, Nylon-12, polymethylmethacylate, aluminum starch octenylsuccinate, lauroyl lysine, calcium starch octenylsuccinate, boron nitride, polymethacrylate, nanoparticles, carboxymethylcellulose, micronized silicone polymers (available from Dow), graphite, silica, ethylcellulose resins (available from Dow), micronized chitin, hydroxyethylcellulose, talc, cationic polymers, silsesquioxanes, starch, cationics such as stearalkonium chloride (i.e., stearyl dimethyl benzyl ammonium chloride), glycols, and combinations thereof. More preferably, the skin texture modulator is Nylon-12.

The skin texture modulator may be present in the compositions in amounts of from about 0.01% (by total weight of the formulation) to about 10% (by total weight of the formulation), more preferably from about 0.1% (by total weight of the formulation) to about 8% (by total weight of the formulation), and still more preferably from about 1% (by total weight of the formulation) to about 7% (by total weight of the formulation), and more preferably is present in an amount of about 5% (by total weight of the formulation).

In one particular embodiment, the skin texture modulator is a nylon. Nylons are a family of thermoplastic, synthetic polymer materials made of repeating units linked by amide bonds. Preferably, the nylon used in the formulations described herein is Nylon-12. Without wishing to be bound to any particular theory, it is believed the Nylon-12 acts to fill in wrinkles and creases in the skin to create smooth skin, without altering the natural skin texture. In one aspect, the skin texture modulator is Nylon-12 and is present in the formulation in an amount of from about 1% (by total weight of the formulation) to about 7% (by total weight of the formulation), and more preferably is present in an amount of about 5% (by total weight of the formulation).

It should be noted that in certain instances a formulation component may have a dual function. For example, Nylon-12 may act as both a coefficient of friction modulator as well as a skin texture modulator. In instances where the formulation comprises Nylon-12, additional coefficient of friction modulators or skin texture modulators other than Nylon-12 may be included in the formulation. However, since Nylon-12 may function as both a coefficient of friction modulator and a skin texture modulator, additional coefficient of friction modulators or skin texture modulators are not required.

The formulations may further comprise a temperature modulator. Skin temperature may vary for different regions of the body, but typically remains between about 32° C. (89.6° F.) and about 35° C. (95° F.). In order to be comfortable, it is preferable for the skin to be at a temperature of about 33° C. (91.4° F.). Advantageously, the formulations of the present disclosure may comprise a temperature modulator to help maintain skin temperature within normal temperature ranges. More particularly, in one aspect, the formulations of the present disclosure will maintain skin temperature at from about 32° C. to about 35° C. for at least about 4 hours, and preferably for at least about 6 hours after application of the formulation to the skin. Skin temperature measurements can be made using any suitable means known in the art. One example of a suitable means of measuring skin temperature is set forth in the examples.

Examples of suitable temperature modulators include insulating agents, warming agents, cooling agents, and combinations thereof. Insulating agents include, for example, mineral oil, glycerol, dimethicone, silicone crosspolymers, petrolatum, water, polysaccharides, hydrogels, waxes, fatty acids, fatty alcohols, and combinations thereof. Preferably, the insulating agent is selected from the group consisting of mineral oil, water, petrolatum, dimethicone, silicone crosspolymers, glycerol, waxes, and combinations thereof. More preferably, the insulating agent is mineral oil.

Suitable warming agents include, for example, equal mixtures of glycerine, glycols, and polyglycols/polyglycerols; SALHEAT (available from Salvona); HOTACT® VBE (vanillyl butyl ether) (available from Lipo); capsaicin; vanillin derivatives; MgCl₂; CaCl₂; zeolite, magnesium sulfate, PEG-7 glyceryl cocoate, and combinations thereof. Preferably, the warming agent is selected from the group consisting of MgCl₂, CaCl₂, zeolite, SALHEAT (available from Salvona), HOTACT® VBE (vanillyl butyl ether) (available from Lipo), capsaicin, vanillin derivatives, and combinations thereof.

Examples of suitable cooling agents include, for example, menthol, menthol derivatives, encapsulated cooling agents such as SALCOOL™ cooling composition (available from Salvona LLC, Dayton, N.J.), menthyl lactate, menthyl salicylate, menthyl acetate, menthyl PCA, menthyl carbinol, methyl linalool, isoeugenol, methyl eugenol, ICE 1500™ cooling sensate (available from Qarôma, Inc., Baytown, Tex.), menthone glycerol ketal, menthoxypropane-1,2-diol, (−)-isopulegol, cubebol, N-substituted p-menthane carboxamides, icilin, mint, mint oils, cucumber, chamomile, aloe, comfrey, anise, sage, carboxamides, ketals, carboxamides, cyclohexanol derivatives, and/or cyclohexyl derivatives. Additional suitable neurosensory components are described in “Cool without Menthol & Cooler than Menthol and Cooling Compounds as Insect Repellents”, John C. Leffingwell, Ph.D., leffingwell.com/cooler_than_menthol.htm, Apr. 19, 2007, which is hereby incorporated by reference to the extent it is consistent (i.e., not in conflict) herewith.

Additionally, cooling agents can consist of evaporative cooling agents such as water; hydrocarbons such as isododecane and isoeicosane; short chain alcohols such as ethanol and n-propanol; small branched chain alcohols such as isopropyl alcohol; fluorinated hydrocarbons such as perfluorodecalin, perfluoroheptane, perfluorohexane, and perfluoromethylcyclohexane; fluorinated alcohols such as C₆-C₁₂ perfluoroalkylethanol and perfluorocyclohexylmethanol; fluorinated ethers such as ethyl perfluorobutyl ether, ethyl perfluoroisobutyl ether, methyl perfluorobutyl ether, methyl perfluoroisobutyl ether, and perfluorohexylethyl dimethylbutyl ether; low molecular weight grades of dimethicone, particularly DOW CORNING® 200 dimethicone fluid 0.65 cst; volatile cyclomethicones such as octamethyl cyclotetrasiloxane, decamethyl cyclopentasiloxane, dodecamethyl cyclohexasiloxane, and tetradecamethyl cycloheptasiloxane. Additionally, phase change compounds which cool due to a change in state on the skin could be used. All of these cooling agents may be used individually or in any combination thereof.

Typically, the temperature modulator will be present in the formulations in an amount of from about 0% (by total weight of the formulation) to about 25% (by total weight of the formulation), more typically from about 0.1% (by total weight of the formulation) to about 15% (by total weight of the formulation), and more typically in an amount from about 1% (by total weight of the formulation) to about 10% (by total weight of the formulation).

The formulations of the present disclosure may further comprise an anti-adherence agent. As noted above, menstrual fluid and other body exudates may wick along the skin and hair and remain attached thereto. This can negatively impact the ability of absorbent products to intake these exudates. Furthermore, a variety of microflora may be found adhered to the surface of skin, including various pathogenic bacteria and yeast which may be associated with numerous ailments, such as skin infections, diaper rash, urinary or vaginal infections, and malodors, among others. An anti-adherence agent may thus be incorporated into the formulations of the present disclosure to prevent or reduce the adherence of menses, fecal material, and/or other body exudates to the skin. Additionally, the anti-adherence agent helps to reduce the adherence of problematic microflora to the skin or mucosa.

Without being bound to a particular theory, it is believed that the anti-adherence agent attaches to the skin through electrical and hydrophobic interaction with the skin and remains tightly bound thereto after deposit. When menstruation and/or defecation occurs, exudates and/or bacteria and enzymes present therein, which typically attach to skin through electrical interactions, are not able to attach to the skin, because many of the binding sites are already occupied with anti-adherence agent. Because electrical and hydrophilic interaction with the bacteria and enzymes and the skin is reduced, much less menstrual fluid, fecal matter, and/or other body exudates remains attached to the skin after menstruation, defecation, urination, and the like. With reduced attachment to the skin, the exudates can be more effectively transported to an absorbent article.

Examples of suitable anti-adherence agents include, for example, alginic acid, β-benzal-butyric acid, botanicals, casein, dextrans (nominally 4000-1000 Da), farnesol, flavones, fucans, galactolipid, high molecular weight kininogen, hyaluronate, inulin, iridoid glycosides, nanoparticles, perlecan, phosphorothioate oligodeoxynucleotides, pluronic surfactants Poloxamer 407, polymethylmethacrylate, pluronic surfactants silicone, sulphated exopolysaccharides, tetrachlorodecaoxide, dimethicone and related compounds, and combinations thereof. Preferably, the anti-adherence agent is selected from the group consisting of dextrans (nominally 4,000 to 10,000 Da), farnesol, flavones, fucans, galactolipid, high molecular weight kininogen, hyaluronate, inulin, iridoid glycosides, nanoparticles, and combinations thereof.

In one particular embodiment, the anti-adherence agent is inulin. Inulins are a group of naturally occurring oligosaccharides, and belong to a class of carbohydrates known as fructans. The inulins are produced by many types of plants and are readily available from commercial vendors. Examples of personal care articles that comprise inulins are described in U.S. Patent App. Publ. No. 2005/0244481, herein incorporated by reference.

In another aspect, the anti-adherence agent is a thermoplastic polymer. Examples of thermoplastic polymers include polymethylmethacrylate, methyl methacrylate crosspolymer, ethylene/acrylate copolymer, polymethylsilsesquioxane, silicone resins, and the like, such as those described in U.S. Patent App. Publ. No. 2005/0129741, herein incorporated by reference.

The thermoplastic polymers may suitably be in the form of a spherical powder or spherical resin. The thermoplastic polymers suitably have an average particle diameter of from about 0.1 micrometers to about 20 micrometers, and suitably from about 0.1 micrometers to about 12 micrometers, and even more suitably from about 0.4 micrometers to about 7 micrometers. Without wishing to be bound to any particular theory, it is believed that polymers having diameters within these ranges act to scatter light and fill wrinkles on the skin better than larger particles which may sit on the skin.

The anti-adherence agent may be present in the formulations in amounts of from about 0.01% (by total weight of the formulation) to about 5% (by total weight of the formulation), more preferably from about 0.1% (by total weight of the formulation) to about 1% (by total weight of the formulation). In one particular embodiment, the anti-adherence agent is inulin and is present in the formulation in an amount of from about 0.1% (by total weight of the formulation) to about 0.25% (by total weight of the formulation), and preferably is present in an amount of about 0.1% (by total weight of the formulation). Advantageously, the anti-adherence agent can additionally assist with reducing viscoelastic properties of menstrual fluid and other body exudates.

In one embodiment, the formulations of the present disclosure may further comprise a treatment agent capable of altering the viscosity, elasticity, and/or fouling effects of fluids having viscoelastic properties, such as menses, mucous, blood products, and feces, among others. Altering the viscoelastic and/or fouling properties of these exudates may help to improve fluid intake, distribution, and absorption properties and decrease leakage of absorbent articles.

In particular, the relatively high viscosity and elasticity of viscoelastic fluids tends to interfere with the absorption and distribution of viscoelastic fluids within absorbent articles, oftentimes resulting in leaking of the absorbent article. Additionally, viscoelastic fluids may result in fouling of absorbent articles. As used herein, “fouling” means the change in permeability of a fluid as it passes through a porous medium. More particularly, fouling is the reduction in permeability that occurs when components of a fluid pass through a porous medium and interact with the material structure, decreasing the inherent permeability of the porous material. Without wishing to be bound to any particular theory, it is believed that fouling by viscoelastic fluids such as menses is likely due to mucin globules present in the fluid. Mucin is a large glycoprotein present in mucus-like fluids that gives the fluids most of their mucous-like properties. Mucin can exist in both a soluble form in the fluid and as mucin globules. Mucin globules are typically in the range of about 50 to about 200 microns in size, and comprise gelled or aggregated mucin molecules. Viscoelastant agents may act in several ways to reduce fouling. For example, it is believed that some viscoelastant agents dissolve the mucin aggregates and significantly reduce the number of these globules, resulting in a reduction in fouling. Other viscoelastant agents may act to reduce the effects of the soluble mucin molecules that produce the elastic or “stringy” quality of menses, but have little or no effect on the mucin globules. Other viscoelastant agents may have both effects.

Thus, in certain embodiments, the formulations of the present disclosure may optionally further comprise a viscoelastic agent that is effective at reducing the viscosity, elasticity, and/or fouling effects of viscoelastic fluids such as menses. Preferably, the viscoelastic agent will reduce both viscosity and elasticity of viscoelastic fluids that come in contact with the viscoelastic agent by at least about 10%, more preferably by at least about 30%, more preferably by at least about 40%, more preferably by at least about 50%, more preferably by at least about 60%, and still more preferably by at least about 70% as compared to untreated viscoelastic fluids, when measured at a temperature of 22° C., a shear rate of 1.0 sec-1, and a frequency of 0.1 Hertz. Additionally, the viscoelastic agent will also preferably reduce the fouling properties of viscoelastic fluids that come in contact with the viscoelastic agent by at least about 20%, more preferably by at least about 40%, and more preferably by at least about 50% as compared to untreated viscoelastic fluids.

It should be recognized that the viscoelastic agents described herein may exert various combinations of effects on viscosity, elasticity, and fouling, depending on the concentration at which they are applied to the substrate.

In one embodiment, the viscoelastic agent is selected from the group consisting of polyethylene glycol laurates, polyethylene glycol lauryl ethers, and combinations thereof. Advantageously, the polyethylene glycol laurates and polyethylene glycol lauryl ethers are capable of reducing both the viscosity and elasticity of viscoelastic fluid. Examples of suitable polyethylene glycol laurates include polyethylene glycol 400 monolaurate, polyethylene glycol 600 monolaurate, polyethylene glycol 1000 monolaurate, polyethylene glycol 4000 monolaurate, polyethylene glycol 600 dilaurate, and combinations thereof. Examples of suitable polyethylene glycol lauryl ethers include polyethylene glycol 600 lauryl ether. Preferably, the polyethylene glycol lauryl ether and/or polyethylene glycol laurate treatment agent is further capable of reducing the fouling properties of viscoelastic fluid. Particularly preferred examples of treatment agents include polyethylene glycol (PEG) 600 lauryl ether and related compounds, polyethylene glycol (PEG) 600 monolaurate and related compounds, and combinations thereof.

In addition to the PEG laurates and PEG lauryl ethers, other polyethylene glycol derivatives may be viscoelastic agents (i.e., are capable of reducing the viscosity and elasticity of viscoelastic fluids) and may be used in the formulations described herein. As used herein, the term “polyethylene glycol derivative” includes any compound comprising a polyethylene glycol moiety. Examples of other suitable PEG derivatives include PEG monostearates such as PEG 200 monostearates and PEG 4000 monostearate; PEG dioleates such as PEG 600 dioleate and PEG 1540 dioleate; PEG monooleates such as PEG 600 monooleate and PEG 1540 monooleate; PEG monoisostearates such as PEG 200 monoisostearate; and PEG 16 octyl phenyl. Particularly preferred polyethylene glycol derivatives for use as viscoelastic agents are those that improve intake time of viscoelastic fluids as well as reduce viscosity and elasticity. Examples of preferred PEG derivatives include PEG 1540 dioleate, PEG 600 monooleate, PEG 1540 monooleate, and PEG 16 octyl phenyl. These PEG derivatives may be used alone or in combination with PEG 600 monolaurate, PEG 600 lauryl ether, and/or other viscoelastic agents.

Other examples of suitable viscoelastic agents include sodium citrate, dextran, cysteine, Glucopon 220UP (available as a 60% (by weight) solution of alkyl polyglycoside in water from Henkel Corporation), Glucopon 425, Glucopon 600, Glucopon 625. Other suitable viscoelastic agents are described in U.S. Pat. No. 6,060,636, herein incorporated by reference in its entirety. Surprisingly, it has been discovered that certain viscoelastic agents that actually increase the fouling effect of viscoelastic fluids when used alone, will in fact improve fouling effects when used in combination with PEG 600 lauryl ether and/or PEG 600 monolaurate. For example, in one embodiment, sodium citrate may be used in combination with PEG 600 monolaurate as a viscoelastic agent. When two or more viscoelastic agents are used in combination, the proportion of each viscoelastic agent present in the formulation is preferably in a ratio of from about 1:2 to about 2:1, and more preferably is about 1:1.

The viscoelastic agent may be used in varying amounts depending on the desired results and application. Typically, the viscoelastic agent is present in the formulation in an amount of from about 0.05% (by total weight of the formulation) to about 25% (by total weight of the formulation), more preferably in an amount of from about 0.1% (by total weight of the formulation) to about 20% (by total weight of the formulation), and still more preferably in an amount of from about 0.5% (by total weight of the formulation) to about 15% (by total weight of the formulation).

The formulations of the present disclosure may further comprise one or more conventional pharmaceutically-acceptable and compatible carrier material. As noted above, the formulations may take a variety of forms including, without limitation, aqueous solutions, gels, balms, lotions, suspensions, creams, milks, salves, ointments, sprays, emulsions, oils, resins, foams, solid sticks, aerosols, and the like. Carrier materials suitable for use in the present disclosure include those well-known for use in the cosmetic and medical arts as a basis for ointments, lotions, creams, salves, aerosols, gels, suspensions, sprays, foams, and the like, and may be used in their art-established levels.

Non-limiting examples of suitable carrier materials include water, emollients, sterols or sterol derivatives, natural and synthetic fats or oils, viscosity enhancers, rheology enhancers, polyols, surfactants, alcohols, esters, silicones, clays, starch, cellulose, and other pharmaceutically acceptable carrier materials. As will be recognized by one skilled in the art, the relative amounts of such components in the formulations of the disclosure will be dictated by the nature of the formulation. The levels can be determined by routine experimentation in view of the disclosure provided herein, and may be present in the formulation in amounts of from about 1% (by total weight of the formulation) to about 99% (by total weight of the formulation).

Thus, in one embodiment, the formulation of the disclosure can optionally include one or more emollient, which typically acts to soften, soothe, and otherwise lubricate and/or moisturize the skin. Suitable emollients that can be incorporated into the formulations include oils such as petrolatum based oils, petrolatum, vegetable based oils, mineral oils, natural or synthetic oils, alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof.

The esters can include, but are not limited to, octyldodecyl neopentanoate, myristyl myristate, cetyl palmitate, stearyl palmitate, cetyl stearate, isopropyl laurate, isopropyl myristate, isopropyl palmitate, and combinations thereof. The fatty alcohols may include, but are not limited to octyldodecanol, lauryl, myristyl, cetyl, stearyl, behenyl alcohol, and combinations thereof. Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof can also suitably be used as emollients.

The formulation may desirably include one or more emollient in an amount of from about 0.01% (by total weight of the formulation) to about 30% (by total weight of the formulation), more desirably from about 0.05% (by total weight of the formulation) to about 25% (by total weight of the formulation), and even more desirably from about 0.10% (by total weight of the formulation) to about 20% (by total weight of the formulation).

Sterol and sterol derivatives which are suitable for use in the formulations of the present disclosure include, but are not limited to cholestol, sitosterol, stigmasterol, ergosterol, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyidecanoate, ergocalciferol, tall oil sterol, soy sterol acetate, lanasterol, soy sterol, avocado sterols, fatty alcohols, and combinations thereof.

The formulation of the disclosure can desirably include sterols, sterol derivatives or mixtures of both sterols and sterol derivatives in an amount of from about 0.01% (by total weight of the formulation) to about 10% (by total weight of the formulation), more desirably from about 0.05% (by total weight of the formulation) to about 5% (by total weight of the formulation), and even more desirably from about 0.1% (by total weight of the formulation) to about 1% (by total weight of the formulation).

The formulations of the disclosure can also include natural fats and oils. As used herein, the term “natural fat or oil” is intended to include fats, oils, essential oils, essential fatty acids, non-essential fatty acids, phospholipids, and combinations thereof. These natural fats and oils can provide a source of essential and non-essential fatty acids to those found in the skin's natural barrier. Suitable natural fats or oils can include citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, maleated soybean oil, meadowfoam oil, palm kernel oil, peanut oil, rapeseed oil, grapeseed oil, safflower oil, sphingolipids, seed almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, sunflower oil, soybean oil, and combinations thereof.

The formulation of the disclosure may desirably include fats and oils in an amount of from about 0.01% (by total weight of the formulation) to about 30% (by total weight of the formulation), more desirably from about 0.05% (by total weight of the formulation) to about 25% (by total weight of the formulation), and even more desirably from about 0.1% (by total weight of the formulation) to about 20% (by total weight of the formulation).

Optionally, one or more viscosity enhancers may be added to the formulation to increase the viscosity, to help stabilize the formulation, such as when the formulation is incorporated into a personal care product, thereby reducing migration of the formulation and improving transfer to the skin. Suitable viscosity enhancers include polyolefin resins, lipophilic/oil thickeners, ethylene/vinyl acetate copolymers, polyethylene, silica, silica silylate, silica methyl silylate, colloidal silicone dioxide, cetyl hydroxy ethyl cellulose, other organically modified celluloses, PVP/decane copolymer, PVM/MA decadiene crosspolymer, PVP/eicosene copolymer, PVP/hexadecane copolymer, clays, carbomers, acrylic based thickeners, polyethylene glycol 600, polyethylene glycols, myristyl alcohol, cetyl alcohol, stearyl alcohol, behenyl alcohol, silicone crosspolymers, polyamide blend, and combinations thereof.

The formulation may desirably include one or more viscosity enhancers in an amount of from about 0.01% (by total weight of the formulation) to about 25% (by total weight of the formulation), more desirably from about 0.05% (by total weight of the formulation) to about 20% (by total weight of the formulation), and even more desirably from about 0.1% (by total weight of the formulation) to about 15% (by total weight of the formulation).

The formulations of the disclosure may optionally further comprise rheology enhancers. Rheology enhancers may help increase the melt point viscosity of the formulation so that the formulation readily remains on the surface of a personal care product and does not substantially migrate into the interior of the product, while substantially not affecting the transfer of the formulation to the skin. Additionally, the rheology enhancers help the formulation to maintain a high viscosity at elevated temperatures, such as those encountered during storage and transportation.

Suitable rheology enhancers include combinations of alpha-olefins and styrene alone or in combination with mineral oil or petrolatum, combinations of di-functional alpha-olefins and styrene alone or in combination with mineral oil or petrolatum, combinations of alpha-olefins and isobutene alone or in combination with mineral oil or petrolatum, ethylene/propylene/styrene copolymers alone or in combination with mineral oil or petrolatum, butylene/ethylene/styrene copolymers alone or in combination with mineral oil or petrolatum, ethylene/vinyl acetate copolymers, polyethylene polyisobutylenes, polyisobutenes, polyisobutylene, dextrin palmitate, dextrin palmitate ethylhexanoate, stearoyl inulin, stearalkonium bentonite, distearadimonium hectorite, and stearalkonium hectorite, styrene/butadiene/styrene copolymers, styrene/isoprene/styrene copolymers, styrene-ethylene/butylene-styrene copolymers, styrene-ethylene/propylene-styrene copolymers, (styrene-butadiene) n polymers, (styrene-isoprene) n polymers, styrene-butadiene copolymers, and styrene-ethylene/propylene copolymers, and combinations thereof. Specifically, rheology enhancers such as mineral oil and ethylene/propylene/styrene copolymers, and mineral oil and butylene/ethylene/styrene copolymers (Versagel blends from Penreco) are particularly preferred. Also, Vistanex (Exxon) and Presperse (Amoco) polymers are particularly suitable rheology enhancers. Another suitable rheology enhancer is RM2051 (INCI: sodium polyacrylate (and) dimethicone (and) cyclopentasiloxane (and) trideceth-6 (and) PEG/PPG-18/18 dimethicone), available from Dow Corning.

The formulation of the present disclosure can suitably include one or more rheology enhancer in an amount of from about 0.01% (by total weight of the formulation) to about 20% (by total weight of the formulation), and more typically about 0.1% (by total weight of the formulation) to about 10% (by total weight of the formulation).

The formulations may further comprise a structurant. The structurant utilized in the formulations described herein help to immobilize the formulation components on the surface of a personal care product to which it is applied. Suitable structurants for use in the formulations disclosed herein include, for example, waxes including animal waxes, vegetable waxes, mineral waxes, synthetic waxes, and polymers. Exemplary structurants include bayberry wax, beeswax, stearyl dimethicone, stearyl trimethicone, C₂₀-C₂₂ dimethicone, C₂₀-C₂₂ trimethicone, C₂₄-C₂₈ dimethicone, C₂₀-C₂₂ trimethicone, C₃₀ alkyl dimethicone, candelilla wax, carnauba, ceresin, cetyl esters, stearyl benzoate, behenyl benzoate, esparto, hydrogenated cottonseed oil, hydrogenated jojoba oil, hydrogenated jojoba wax, hydrogenated microcrystalline wax, hydrogenated rice bran wax, japan wax, jojoba butter, shea butter, cocoa butter, jojoba esters, jojoba wax, lanolin wax, microcrystalline wax, mink wax, motan acid wax, motan wax, ouricury wax, cerasin wax, ozokerite paraffin, PEG-6 beeswax, PEG-8 beeswax, rezowax, rice bran wax, shellac wax, spent grain wax, spermaceti wax, synthetic spermaceti wax, synthetic beeswax, synthetic candelilla wax, synthetic carnuba wax, synthetic japan wax, synthetic jojoba wax, C₁₄-C₂₈ fatty acid ethoxylates and C₁₄-C₂₈ fatty ethers, C₁₄-C₂₈ fatty alcohols, C₁₄-C₂₈ fatty acids, polyethylene, oxidized polyethylene, ethylene-alpha olefin copolymers, ethylene homopolymers such as Petrolite EP copolymers from Baker Hughes Inc., (Sugar Land Tex.), C₁₈-C₄₅ olefins, poly alpha olefins such as Vybar Polymers from Baker Hughes Inc. or Okerin Polymers from Honeywell Specialty Chemicals, (Duluth, Ga.), hydrogenated vegetable oils, C₂₄-C₃₄ α-olefin and polyethylene, polyhydroxy fatty acid esters, polyhydroxy fatty acid amides, ethoxylated fatty alcohols and esters of C₁₂-C₂₈ fatty acids, C₁₂-C₂₈ fatty alcohols, ozokerite and alkyl silicone, petrolatum USP, ethylene/vinyl acetate copolymer, stearyl behenate, stearyl alcohol, isopropyl palmitate, fumed silica, celluloses, gums, clays, carbomers, acrylate derivatives, and combinations thereof.

The structurant may be included in the formulations in amounts of from about 1% (by total weight of the formulation) to about 75% (by total weight of the formulation), more typically from about 10% (by total weight of the formulation) to about 50% (by total weight of the formulation), desirably from about 20% (by total weight of the formulation) to about 40% (by total weight of the formulation).

In one embodiment, the formulations may comprise water. For instance, where the formulation is a wetting composition or liquid formulation, such as described herein for use with a wet wipe, the formulation will typically comprise water. The formulations can suitably comprise water in an amount of from about 0.1% (by total weight of the formulation) to about 99% (by total weight of the formulation), more preferably from about 1% (by total weight of the formulation) to about 90% (by total weight of the formulation), and still more preferably from about 20% (by total weight of the formulation) to about 85% (by total weight of the formulation).

The formulation may further comprise fragrances. Any suitable fragrance may be used. Typically, the fragrance is present in the formulation in an amount of from about 0.01% (by total weight of the formulation) to about 5% (by total weight of the formulation). In one preferred embodiment, the fragrance will have a clean, fresh, and/or neutral scent to create an appealing delivery vehicle for the end consumer.

The formulation may further comprise a chelating agent. Chelating agents may act to enhance preservative efficacy, and bind metals that could discolor the formulation or hinder formulation stability. Suitable chelating agents include, for example, disodium ethylenediamine tetraacetic acid (EDTA), commercially available from the Dow Chemical Company under the name VERSENE Na₂. The chelating agent may be present in the formulation in an amount of from about 0.01% (by total weight of the formulation) to about 10% (by total weight of the formulation), and more typically in an amount of about 0.2% (by total weight of the formulation).

In other embodiments, the compositions may optionally comprise a pH adjuster, such as an acid or alkali material. Any suitable acid or alkali material may be used, such as malic acid, potassium hydroxide, and the like. Preferably, the pH adjuster is included in the formulation in amounts suitable to adjust the formulation to the desired pH. Typically, the pH of the formulation will be from about 4 to about 9, more typically will be from about 4.5 to about 7, and more typically is about 6.0.

In one particular embodiment, the formulation may comprise from about 1% (by total weight of the formulation) to about 15% (by total weight of the formulation) of a moisturizer, from about 0.01% (by total weight of the formulation) to about 10% (by total weight of the formulation) of a coefficient of friction modulator, from about 0.01% (by total weight of the formulation) to about 10% (by total weight of the formulation) of a skin texture modulator, and from about 0.01% (by total weight of the formulation) to about 10% (by total weight of the formulation) of an elasticity modulator. Optionally, the formulation may further comprise from about 0% (by total weight of the formulation) to about 25% (by total weight of the formulation) of a temperature modulator, from about 0.01% (by total weight of the formulation) to about 5% (by total weight of the formulation) of an anti-adherence agent, and/or from about 5% (by total weight of the formulation) to about 15% (by total weight of the formulation) of a viscoelastic agent.

In other aspects, the formulation may further optionally comprise from about 0.01% (by total weight of the formulation) to about 25% (by total weight of the formulation) of a viscosity enhancer, from about 1% (by total weight of the formulation) to about 75% (by total weight of the formulation) of a structurant, from about 0.01% (by total weight of the formulation) to about 5% (by total weight of the formulation) of a fragrance, or combinations thereof.

In one particular embodiment, the formulation may comprise from about 1% (by total weight of the formulation) to about 15% (by total weight of the formulation) of glycerin, from about 0.01% (by total weight of the formulation) to about 10% (by total weight of the formulation) of dimethicone, from about 0.01% (by total weight of the formulation) to about 10% (by total weight of the formulation) of Nylon-12, and from about 0.01% (by total weight of the formulation) to about 10% (by total weight of the formulation) of Vitamin E acetate. Optionally, the formulation may further comprise from about 0% (by total weight of the formulation) to about 25% (by total weight of the formulation) of a temperature modulator such as mineral oil, and/or from about 0.01% (by total weight of the formulation) to about 5% (by total weight of the formulation) of an anti-adherence agent such as inulin, and/or from about 5% (by total weight of the formulation) to about 15% (by total weight of the formulation of a viscoelastic agent such as polyethylene glycol 600 monolaurate or polyethylene glycol 600 lauryl ether.

As noted above, the formulations described herein may be applied to the skin and/or mucosa of a user. In certain aspects, the formulations may be applied to help reduce or prevent the adherence of body exudates and problematic microflora to the skin and hair, and to reduce the occurrence of wicking of body exudates. Advantageously, the formulation can also serve to maximize the transport of the exudates into an absorbent article.

The formulations may be applied using any suitable means including, for example, wipes, absorbent articles, sprays, lotions, washes, foams, films, and the like. For instance, a cream, foam, film, lotion, gel, ointment, paste or the like may be spread on the desired surface and gently rubbed in. In other embodiments, the formulation is incorporated into a wet wipe or dry wipe formulation and applied using a wet wipe or dry wipe. In still other embodiments, the formulation may be incorporated into an absorbent article such that the formulation contacts the skin of a user and is transferred to the skin when the absorbent article is worn.

Thus, in one aspect, the formulations may be incorporated into or onto a substrate such as a wipe substrate, an absorbent substrate, a fabric or cloth substrate, or a tissue substrate, among others. For example, the formulations may be incorporated into personal care products, such as wipes, absorbent articles, bath tissues, cloths, gloves, and the like. More particularly, the formulations may be incorporated into wipes such as wet wipes, hand wipes, face wipes, cosmetic wipes, household wipes, dry wipes, and the like, to improve skin cleanliness and health. The formulations may also be incorporated into absorbent articles, such as diapers, training pants, adult incontinence products, feminine hygiene products, and the like. In one preferred embodiment, the formulation is a liquid formulation that may be used in combination with a wipe substrate to form a wet wipe or may be a wetting composition for use in combination with a dispersible wet wipe. Dispersible wet wipes are described in, for example, U.S. Published Patent App. Serial No. 2007/0141936, herein incorporated by reference.

Although discussed primarily in terms of a liquid formulation for use with a wet wipe, it should be understood that the formulations described herein can also be used in combination with dispersible wet wipes, dry wipes, and other personal care products, such as described above. For instance, the formulations of the present disclosure may be applied to a wipe substrate or personal care product using a variety of techniques, such as, for example, spraying slot coating, gravure coating, ink jet printing and the like. The formulation can be applied to one side or both sides of a wipe in any number of patterns.

Materials suitable for the substrate of the wipe are well known to those skilled in the art, and are typically made from a fibrous sheet material, which may be either woven or nonwoven. For example, the wipes incorporating the formulations described herein to improve skin health may include nonwoven fibrous sheet materials, which include meltblown, coform, air-laid, bonded-carded web materials, hydroentangled materials, spunlace, and combinations thereof. Such materials can be comprised of synthetic or natural fibers, or a combination thereof. Typically, wipes define a basis weight of from about 25 to about 120 grams per square meter and desirably from about 40 to about 90 grams per square meter.

In a particular embodiment, the wipes incorporating the formulations described herein comprise a coform basesheet of polymeric microfibers and cellulosic fibers having a basis weight of from about 60 to about 80 grams per square meter and desirably about 75 grams per square meter. Such coform basesheets are manufactured generally as described in U.S. Pat. No. 4,100,324, which is incorporated by reference. Typically, such coform basesheets comprise a gas-formed matrix of thermoplastic polymeric meltblown microfibers, such as, for example, polypropylene microfibers, and cellulosic fibers, such as, for example, wood pulp fibers.

The relative percentages of the polymeric microfibers and cellulosic fibers in the coform basesheet can vary over a wide range depending upon the desired characteristics of the wet wipes. For example, the coform basesheet may comprise from about 20 to about 100 weight percent, desirably from about 20 to about 60 weight percent, and more desirably from about 30 to about 40 weight percent of the polymeric microfibers based on the dry weight of the coform basesheet being used to provide the wipes.

Alternatively, the wipes incorporating the formulations described herein can comprise a composite, which includes multiple layers of materials such as those described in U.S. Pat. No. 6,028,018, which is incorporated by reference. For example, the wipes may include a three layer composite, which includes an elastomeric film or meltblown layer between two coform layers as described above. In such a configuration, the coform layers may define a basis weight of from about 15 to about 30 grams per square meter and the elastomeric layer may include a film material such as a polyethylene metallocene film.

As mentioned above, one type of wipe suitable for use in combination with the formulations described herein to improve skin health and cleanliness include wet wipes, which contain a liquid solution or formulation. The liquid can be any solution, which can be absorbed into the wet wipe basesheet and may include any suitable components, which provide the desired wiping properties. For example, the components may include water, emollients, surfactants, fragrances, preservatives, organic or inorganic acids, chelating agents, pH buffers, or combinations thereof as are well known to those skilled in the art. Further, the liquid may also contain lotions, medicaments, dyes, and/or antimicrobials. The wipes may also contain the components that comprise the skin health and cleanliness formulations described herein, such as coefficient of friction modulators, moisturizers, elasticity modulators, skin texture modulators, temperature modulators, anti-adherence agents, viscoelastants, and the like. The skin health and cleanliness formulations of the present disclosure may be absorbed into the wipe and/or present in the liquid wet wipe formulation.

The amount of liquid contained within each wet wipe may vary depending upon the type of material being used to provide the wet wipe, the type of liquid being used, the type of container being used to store the wet wipes, and the desired end use of the wet wipe. Generally, each wet wipe can contain from about 150 to about 600 weight percent and desirably from about 250 to about 450 weight percent liquid based on the dry weight of the wipe for improved wiping. In a particular aspect, the amount of liquid contained within the wet wipe is from about 300 to about 400 weight percent and desirably about 330 weight percent based on the dry weight of the wet wipe. If the amount of liquid is less than the above-identified ranges, the wet wipe may be too dry and may not adequately perform. If the amount of liquid is greater than the above-identified ranges, the wet wipe may be oversaturated and soggy and the liquid may pool in the bottom of the container holding the wet wipes.

Each wet wipe is generally rectangular in shape and may have any suitable unfolded width and length. For example, the wet wipe may have an unfolded length of from about 2.0 to about 80.0 centimeters and desirably from about 10.0 to about 25.0 centimeters and an unfolded width of from about 2.0 to about 80.0 centimeters and desirably from about 10.0 to about 25.0 centimeters. Typically, each individual wet wipe is arranged in a folded configuration and stacked one on top of the other to provide a stack of wet wipes. Such folded configurations are well known to those skilled in the art and include c-folded, z-folded, quarter-folded configurations and the like. The stack of folded wet wipes may be placed in the interior of a container, such as a plastic tub, to provide a package of wet wipes for eventual sale to the consumer. Alternatively, the wet wipes may include a continuous strip of material which has perforations between each wipe and which may be arranged in a stack or wound into a roll for dispensing.

As noted above, a personal care product, such as a wipe or tissue, may be used to apply the formulation to the skin. In this embodiment, the wipe containing the formulation is contacted with the skin of a wearer, and the formulation is transferred to the skin. The wipe may be used prior to or during menstruation and prior to defecation, urination, and the like, to reduce or prevent the adherence of body exudates to the skin of a user, to reduce or prevent the wicking of the exudate along the body, and to promote fluid transport from the body to the absorbent product. Alternately, or in addition, the wipe may be used during or following menstruation, defecation, urination, and the like, to assist in cleaning the skin and removing body exudates therefrom.

In another embodiment, personal care products which comprise the formulations described herein for improving skin health and cleanliness can include absorbent articles. As used herein, the phrase “absorbent article” generally refers to devices which absorb and contain body fluids, and more specifically, refers to devices which are placed against or near the skin to absorb and contain the various fluids discharged from the body. Examples of absorbent articles include, without limitation, diapers, training pants, adult incontinence garments, feminine napkins, paper towels, tampons, interlabial pads, facial tissue, wound management products, and bath tissue. Materials and methods for making such absorbent products are well known to those skilled in the art. When utilized in their intended manner, the absorbent personal care products including the formulation described herein contact the skin such that the formulation is contacted with the skin of the article's wearer. In this manner, the formulation may be transferred to the skin, to help improve skin health and cleanliness.

Typically, the formulation is present on the absorbent article in an add on-amount of from about 0.1% (by weight of the treated substrate) to about 25% (by weight of the treated substrate), more preferably in an amount of from about 0.1% (by weight of the treated substrate) to about 20% (by weight of the treated substrate), and still more preferably in an amount of from about 3% (by weight of the treated substrate) to about 12% (by weight of the treated substrate).

The construction and materials used in conventional absorbent articles vary widely and are well known to those of skill in the art. The invention has particular usefulness for feminine care articles and, for purposes of illustration and description only, embodiments of feminine care articles according to the disclosure, in particular sanitary napkins, are referenced herein. However, it should be appreciated that the disclosure is in no way limited to sanitary napkins in particular, or to feminine care articles in general.

Disposable absorbent articles such as, for example, many of the feminine care absorbent products, can include a liquid pervious topsheet (also referred to herein as a cover or body contact layer), a substantially liquid impervious backsheet joined to the topsheet, and an absorbent core positioned and held between the topsheet and the backsheet. The topsheet is operatively permeable to the liquids that are intended to be held or stored by the absorbent article, and the backsheet may be substantially impermeable or otherwise operatively impermeable to the intended liquids. The absorbent article may also include other components, such as liquid wicking layers, liquid intake layers, liquid distribution layers, transfer layers, barrier layers, and the like, as well as combinations thereof. Disposable absorbent articles and the components thereof can operate to provide a body-facing surface and a garment-facing surface. As used herein, the body-facing or bodyside surface means that surface of the article or component which is intended to be disposed toward or placed adjacent to the body of the wearer during ordinary use, while the outward, outward-facing or garment-side surface is on the opposite side, and is intended to be disposed to face away from the wearer's body during ordinary use. Such outward surface may be arranged to face toward or placed adjacent to the wearer's undergarments when the absorbent article is worn. Suitable absorbent articles are described in U.S. Patent Application No. 2004/0186448, herein incorporated by reference in its entirety.

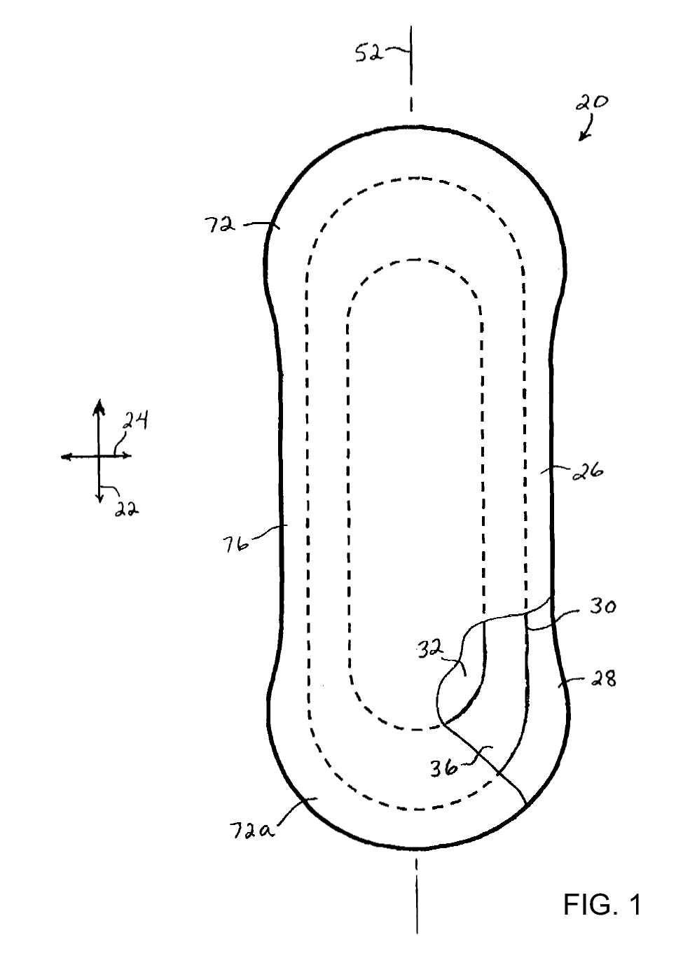

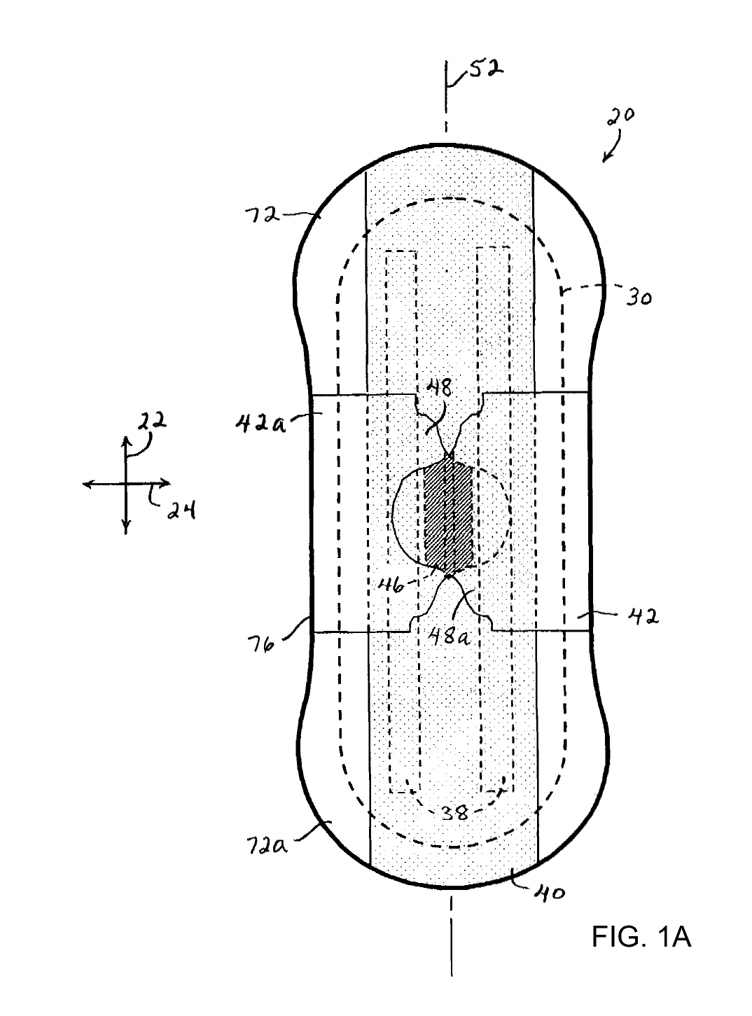

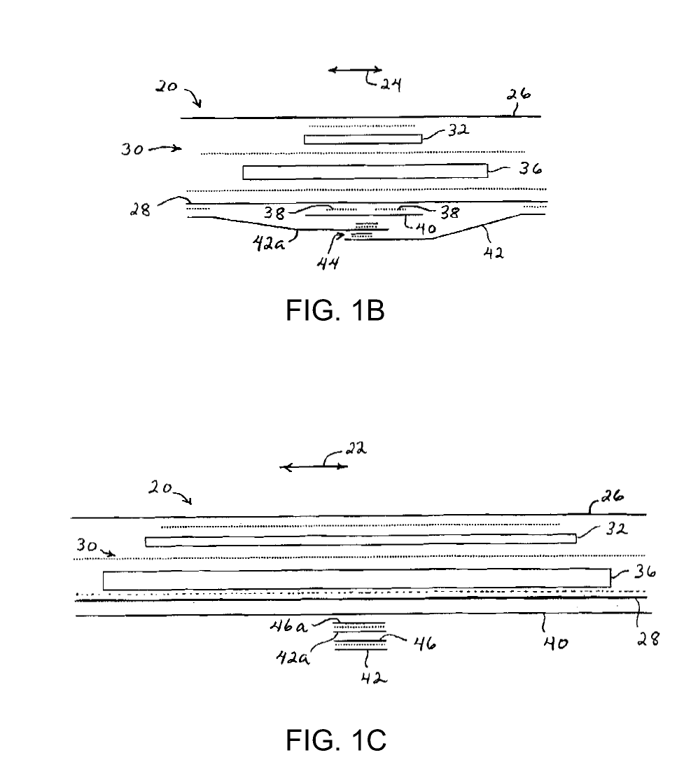

FIGS. 1 through 1C, illustrate an example of a suitable article, such as the representatively shown feminine care article, which is configured to incorporate the present invention. The feminine care article can, for example, be a feminine care pad or napkin 20, and the article can have a lengthwise longitudinal direction 22, a transverse, laterally extending, cross-direction 24, first and second longitudinally opposed end portions 72 and 72a, and an intermediate portion 76 located between the end portions. As representatively shown, the longitudinal dimension of the article is relatively larger than the lateral dimension of the article. The article 20 can include a topsheet or cover 26, a backsheet (also referred to herein as a baffle) 28, and an absorbent structure 30 positioned between the cover and baffle. In a particular aspect, the absorbent structure 30 can at least include an intake layer 32 and a shaping layer 36. In other aspects, the intake and shaping layers can have configurations of absorbent capacities, configurations of densities, configurations of basis weights and/or configurations of sizes which are selectively constructed and arranged to provide desired combinations of liquid intake time, absorbent saturation capacity, absorbent retention capacity, z-directional liquid distribution along the thickness dimension of the article, shape maintenance, and aesthetics.

The cover 26 may include a layer constructed of any operative material, and may be a composite material. For example, the cover layer can include a woven fabric, a nonwoven fabric, a polymer film, a film-fabric laminate or the like, as well as combinations thereof. Examples of a nonwoven fabric include spunbond fabric, meltblown fabric, coform fabric, a carded web, a bonded-carded-web, a bicomponent spunbond fabric or the like as well as combinations thereof. For example, the cover layer can include a woven fabric, a nonwoven fabric, a polymeric film that has been configured to be operatively liquid-permeable, or the like, as well as combinations thereof. Other examples of suitable materials for constructing the cover layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable cover layer material can include a bonded-carded-web composed of polypropylene and polyethylene, such as has been used as a cover stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. In a desired arrangement, the cover layer 26 can be configured to be operatively liquid-permeable with regard to the liquids that the article is intended to absorb or otherwise handle. The operative liquid-permeability may, for example be provided by a plurality of pores, perforations, apertures or other openings, as well as combinations thereof, that are present or formed in the cover layer. The apertures or other openings can help increase the rate at which bodily liquids can move through the thickness of the cover layer and penetrate into the other components of the article (e.g. into the absorbent structure 30). The selected arrangement of liquid-permeability is desirably present at least on an operative portion of the cover layer that is appointed for placement on the body-side of the article. The cover layer 26 can provide comfort and conformability, and can function to direct bodily exudates away from the body and toward the absorbent structure 30. In a desired feature, the cover layer 26 can be configured to retain little or no liquid in its structure, and can be configured to provide a relatively comfortable and non-irritating surface next to the body-tissues of a female wearer. The cover layer 26 can be constructed of any material which is also easily penetrated by bodily fluids that contact the surface of the cover layer.

The cover 26 may be maintained in secured relation with the absorbent structure 30 by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The cover 26 typically extends over the upper, bodyside surface of the absorbent structure, but can alternatively extend around the article to partially or entirely, surround or enclose the absorbent structure. Alternatively, the cover 26 and the baffle 28 can have peripheral margins which extend outwardly beyond the terminal, peripheral edges of the absorbent structure 30, and the extending margins can be joined together to partially or entirely, surround or enclose the absorbent structure.

The baffle 28 may include a layer constructed of any operative material, and may or may not have a selected level of liquid-permeability or liquid-impermeability, as desired. In a particular configuration, the backsheet or baffle 28 may be configured to provide an operatively liquid-impermeable baffle structure. The baffle may, for example, include a polymeric film, a woven fabric, a nonwoven fabric or the like, as well as combinations or composites thereof. For example, the baffle may include a polymer film laminated to a woven or nonwoven fabric. In a particular feature, the polymer film can be composed of polyethylene, polypropylene, polyester or the like, as well as combinations thereof. Additionally, the polymer film may be micro-embossed, have a printed design, have a printed message to the consumer, and/or may be at least partially colored. Desirably, the baffle 28 can operatively permit a sufficient passage of air and moisture vapor out of the article, particularly out of an absorbent (e.g. storage or absorbent structure 30) while blocking the passage of bodily liquids. An example of a suitable baffle material can be a breathable, microporous film, such as a HANJIN Breathable Baffle available from Hanjin Printing, Hanjin P&C Company Limited, a business having offices located in Sahvon-li.Jungan-mvu.Kongiu-City, Chung cheong nam-do, Republic of South Korea. The baffle material is a breathable film, which is white in color, dimple embossed and contains: 47.78% calcium carbonate, 2.22% TiO₂, and 50% polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable baffle material can include a closed cell polyolefin foam. For example, a closed cell polyethylene foam may be employed. Still another example of a baffle material would be a material that is similar to a polyethylene film which is used on commercially sold KOTEX brand pantiliners, and is obtainable from Pliant Corporation, a business having offices located in Schaumburg, Ill., USA.

The structure of the absorbent body 30 can be operatively configured to provide a desired level of absorbency or storage capacity. More particularly, the absorbent body can be configured to hold a liquid, such as urine, menses, other complex liquid or the like, as well as combinations thereof. As representatively shown, the absorbent body can include a matrix of absorbent fibers and/or absorbent particulate material, and the absorbent fiber can include natural and/or synthetic fiber.

The absorbent structure 30 may also include superabsorbent material. Superabsorbent materials suitable for use in the present invention are known to those skilled in the art, and may be in any operative form, such as particulate form. Generally stated, the superabsorbent material can be a water-swellable, generally water-insoluble, hydrogel-forming polymeric absorbent material, which is capable of absorbing at least about 20, desirably about 30, and possibly about 60 times or more its weight in physiological saline (e.g. saline with 0.9 wt % NaCl). The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company and Evonik Degussa. The superabsorbent material may desirably be included in an appointed storage or retention portion of the absorbent system, and may optionally be employed in other components or portions of the absorbent article.

As representatively shown, the absorbent body 30 of the selected article can comprise a composite structure having a selected plurality of strata or layers. With reference to FIGS. 1 through 1C, for example, the absorbent composite can include an intake layer 32 and an absorbent shaping layer 36, as well as any other desired components, arranged in any operative combination. As representatively shown, the structure of the absorbent body can include an absorbent pad, shaping layer 36 which is positioned between the cover 26 and the baffle 28, and can include an intake layer 32 which is positioned between the cover 26 and the shaping layer 36.

In a particular aspect, the article 20 can include a top, bodyside intake layer 32 which is sized and placed to more effectively operate in a target area of the absorbent body 30 where liquids are more likely to be introduced into the article. The material of the intake layer can be configured to provide desired liquid-intake properties, substantially without consideration for delivering shaping properties. For example, the configuration of the intake layer need not include properties that are configured to prevent bunching and twisting of the article, particularly the absorbent structure, during ordinary wear.

The intake layer can include material that is configured to quickly absorb and pull liquid away from the body. Accordingly, the intake layer 32 can provide the function of liquid intake and can also provide the functions of liquid distribution, spreading, temporary storage and liquid retention. The intake layer may include natural fibers, synthetic fibers, superabsorbent materials, a woven fabric; a nonwoven fabric; a wet-laid fibrous web; a substantially unbonded airlaid fibrous web; an operatively bonded, stabilized-airlaid fibrous web; or the like, as well as combinations thereof. Additionally, the absorbent body may include one or more components that can modify menses or intermenstrual liquid.

In a particular arrangement, the intake layer can be a thermally-bonded, stabilized airlaid fibrous web (e.g. Concert code 175.1020) available from Concert Fabrication, a business having offices located in Gatineaux, Quebec, Canada. The intake layer may optionally be provided by a similar, stabilized airlaid fibrous web available from Buckeye Technologies, Inc., a business having offices located in Memphis, Tenn., U.S.A.

In a desired feature, the intake layer 32 can have a relatively lower basis weight, as compared to the bottom (garment-side) retention/shaping layer 36. Optionally, the basis weight of the intake layer may be equal or similar to the basis weight of the shaping layer. In another feature, the intake layer 32 can have a lower density (e.g., be more lofty), as compared to the retention/shaping layer 36.

In a particular aspect, the basis weight of the intake layer 32 can be at least a minimum of about 30 g/m² The basis weight of the intake layer can alternatively be at least about 100 g/m², and can optionally be at least about 120 g/m² to provide improved performance. In other aspects, the basis weight of the intake layer can be up to a maximum of about 250 g/m², or more. The basis weight of the intake layer can alternatively be up to about 200 g/m², and can optionally be up to about 175 g/m² to provide improved effectiveness.

In another aspect, the density of the intake layer 32 can be at least a minimum of about 0.01 g/cm³. The intake layer density can alternatively be at least about 0.02 g/cm³, and can optionally be at least about 0.04 g/cm³ to provide improved performance. In still other aspects, the intake layer density can be up to a maximum of about 0.1 g/cm³, or more. The intake layer density can alternatively be up to about 0.09 g/cm³, and can optionally be up to about 0.08 g/cm³ to provide improved effectiveness. In a desired arrangement, the density of the intake layer can be about 0.06 g/cm³.

A particular feature can include an intake layer 32 which includes fibers that can provide an intake layer that is relatively more “hydrophilic” than the shaping layer 36. Still another feature can include an intake layer wherein at least an operative portion of the fibers have been semi-treated by incorporating a debonding agent to improve opening and fiberization during the manufacturing process. Other suitable intake layer properties are described in U.S. Patent Application No. 2004/0186448.

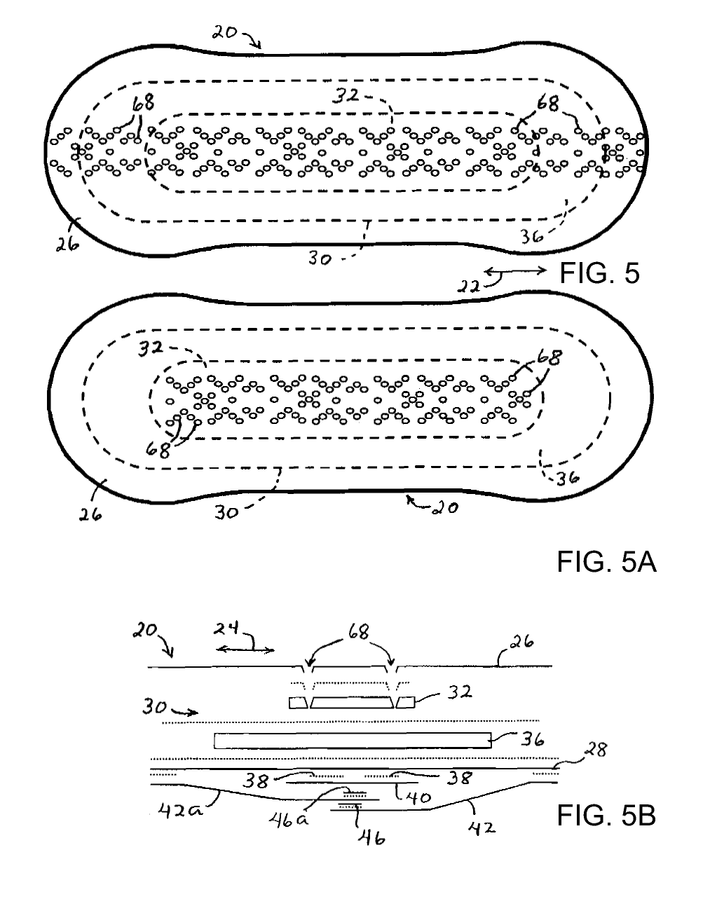

The top intake layer 32 may have any operative shape and/or design. For example, the intake layer may include a single piece of material, or multiple pieces of material, such as multiple strips of material. In addition, the intake layer 32 may include holes or apertures 68 (e.g. FIGS. 5 through 5B) to better provide desired liquid-intake properties. The apertures may extend partially or completely through the z-directional thickness of the intake layer 32, as desired.

The shaping layer 36 can provide the functions of liquid storage and retention, liquid distribution, liquid spreading and shape maintenance. The shaping layer may include natural fibers, synthetic fibers, superabsorbent materials, a woven fabric; a nonwoven fabric; a wet-laid fibrous web; a substantially unbonded airlaid fibrous web; an operatively bonded, stabilized-airlaid fibrous web; or the like, as well as combinations thereof. Additionally, the shaping layer may include one or more components that can modify the menses or intermenstrual liquid.

In a particular arrangement, the shaping layer can be a thermally-bonded, stabilized airlaid fibrous web available from Concert Fabrication (Concert code 225.1021), a business having offices located in Gatineaux, Quebec, Canada (e.g. Concert code 225.1021). The shaping layer 36 may optionally be provided by a similar, stabilized airlaid fibrous web available from Buckeye Technologies, Inc., a business having offices located in Memphis, Tenn., U.S.A.

The shaping layer can have a higher basis weight, as compared to the intake layer 32, but may optionally have a similar or equal basis weight. In another feature, the density of the retention/shaping layer 36 can be greater than that of the intake layer 32, and may include a density gradient through the material of the intake layer (e.g. with higher densities positioned relatively closer to the bottom, garment-side of the article). The equal or greater basis weight and higher density of the shaping layer 36 can result in a relatively stiffer material in the bottom retention/shaping layer 36, as compared to the top intake layer 32. The configuration of the shaping layer 36 can better promote liquid transfer to the baffle-side of the article, away from the wearer's skin, and can decrease the likelihood of liquid rewet or flowback to the wearer's skin.

In a particular aspect, the basis weight of the shaping layer 36 can be at least a minimum of about 100 g/m². The shaping layer basis weight can alternatively be at least about 130 g/m², and can optionally be at least about 165 g/m² to provide improved performance. In other aspects, the basis weight of the shaping layer can be up to a maximum of about 400 g/m², or more. The shaping layer basis weight can alternatively be up to about 350 g/m², and can optionally be up to about 325 g/m² to provide improved effectiveness. In a desired configuration, the shaping layer basis weight can be about 225 g/m².

In a further aspect, the density of the shaping layer 36 can be at least a minimum of about 0.06 g/cm³. The shaping layer density can alternatively be at least about 0.07 g/cm³, and can optionally be at least about 0.08 g/cm³ to provide improved performance. In other aspects, the density of the shaping layer can be up to a maximum of about 0.3 g/cm³, or more. The shaping layer density can alternatively be up to about 0.2 g/cm³, and can optionally be up to about 0.16 g/cm³ to provide improved effectiveness. In a desired arrangement, the density of the shaping layer 36 can be about 0.12 g/cm³. Other suitable properties of shaping layer materials are described in U.S. Patent Application No. 2004/0186448.

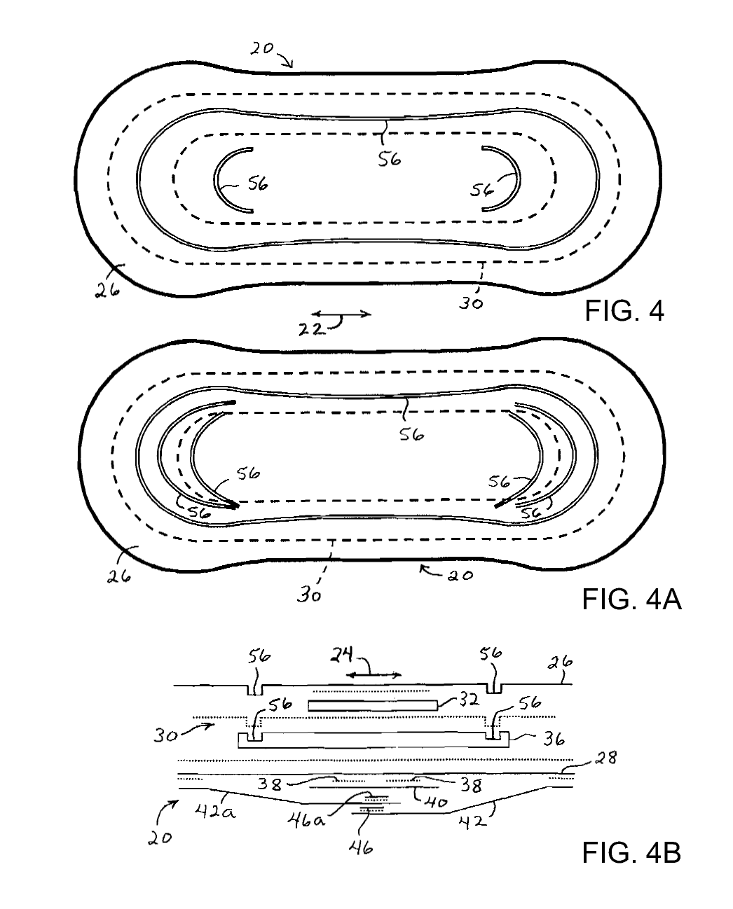

In optional arrangements, the article 20 may include additional components or component layers, as desired. For example, a transfer layer may be positioned between the intake layer 32 and the shaping layer 36. In another feature, the article may include any desired pattern of embossments 56 (e.g. FIGS. 4 through 4B) formed into at least the bodyside surface of the article. The embossing can deform the bodyside of the cover and can deform selected portions of the absorbent body 30 to provide operative channel regions that can help block, direct or otherwise control a desired movement of liquids along the bodyside surface of the article. The embossing can also provide an aesthetic benefit to the consumer, and a visual cue regarding fit and leakage protection. In particular arrangements, the embossments can be positioned generally adjacent the perimeter edges of the absorbent body 30. In other aspects, the embossments can be configured to provide a regular or irregular pattern having one or more channels which are distributed in a symmetrical or asymmetrical array, as desired.

The article 20 can include a system of side-panel or wing portions 42 which can be integrally connected to appointed sections of the side regions along the intermediate portion of the article. For example, the side-panels or wings can be separately provided members that are subsequently attached or otherwise operatively joined to the intermediate portion of the article 20 (e.g. FIGS. 1 through 1C).

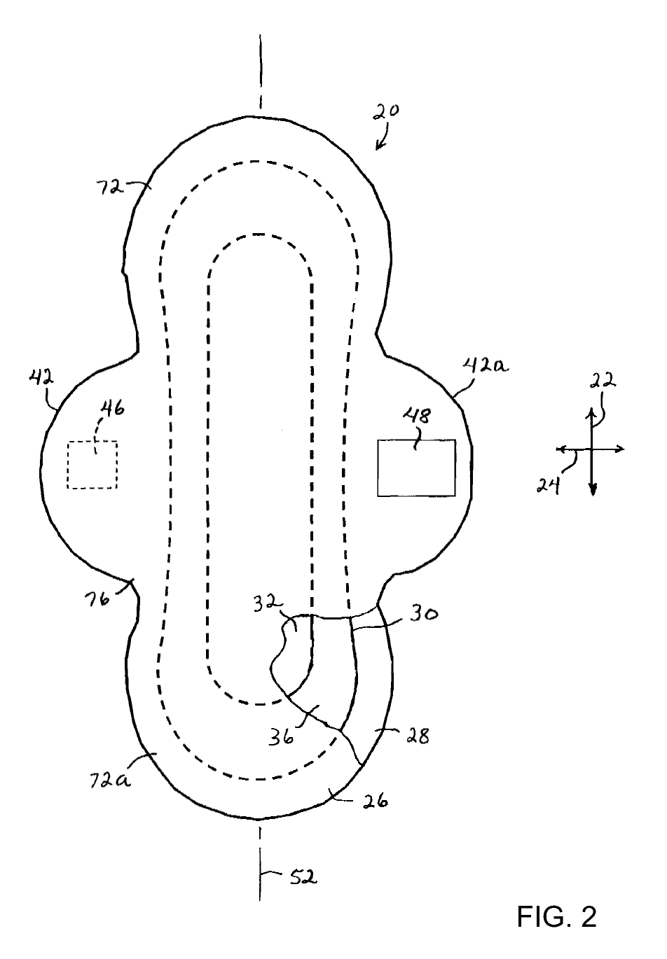

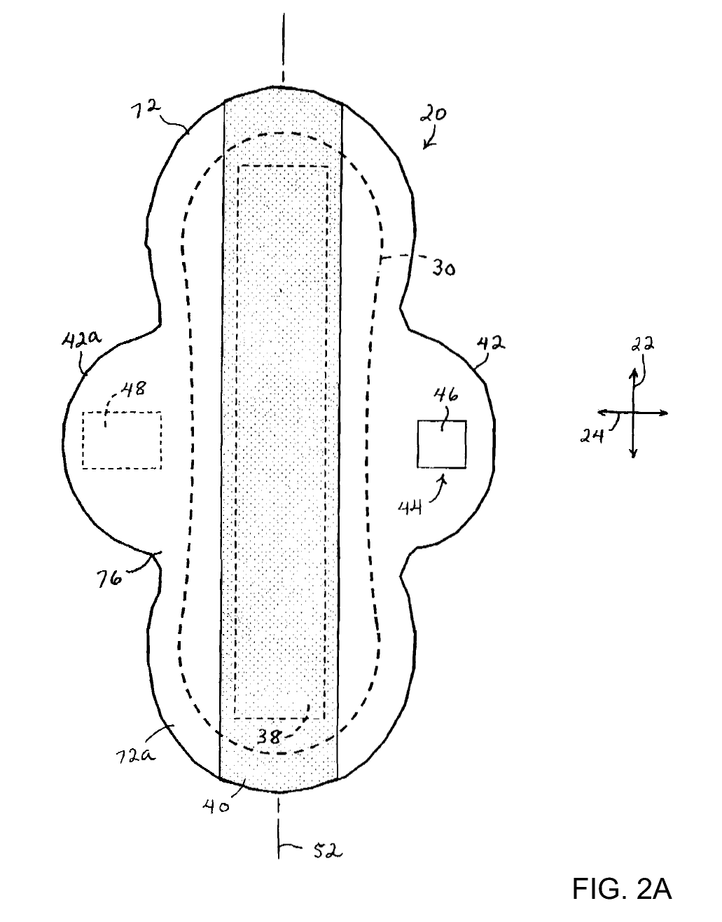

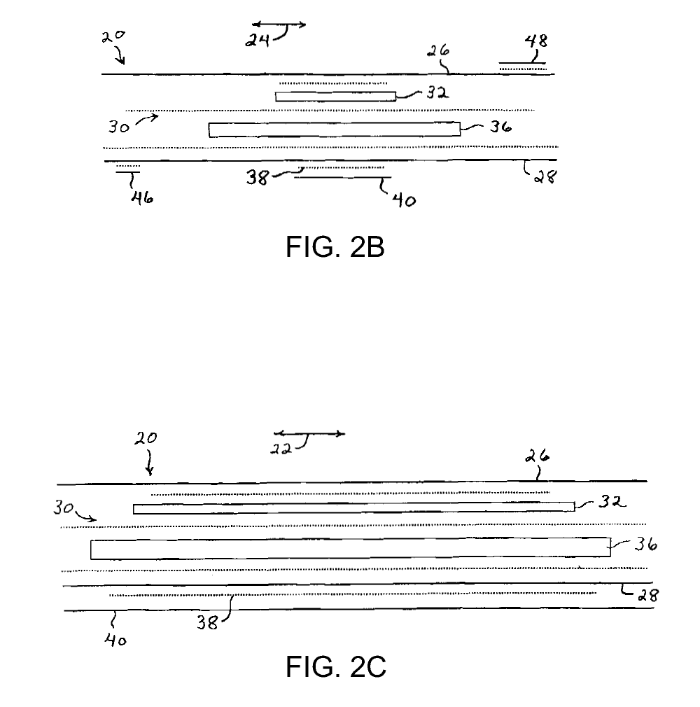

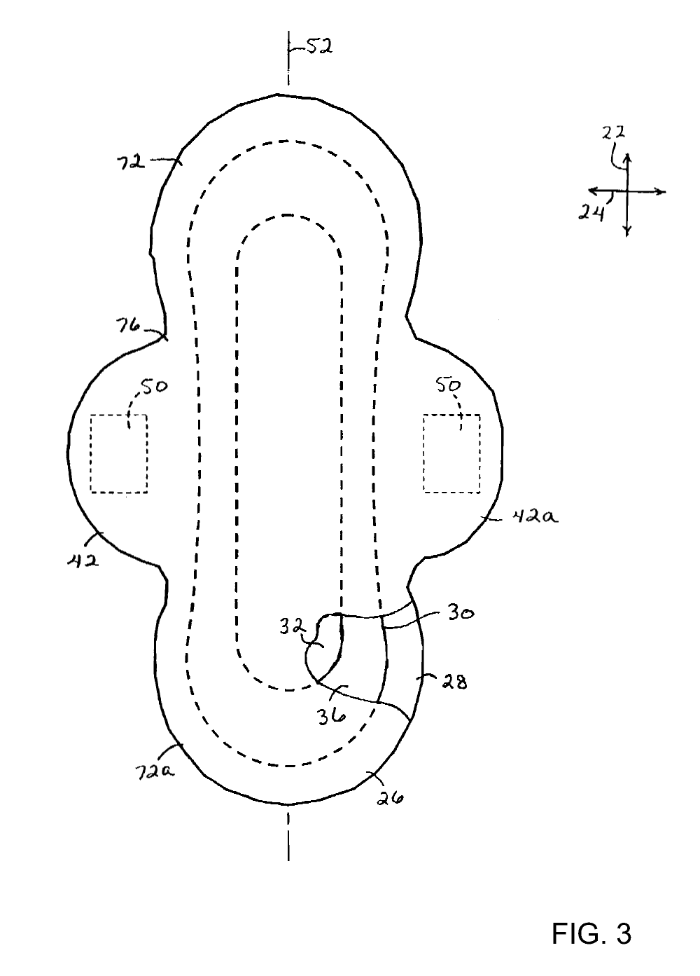

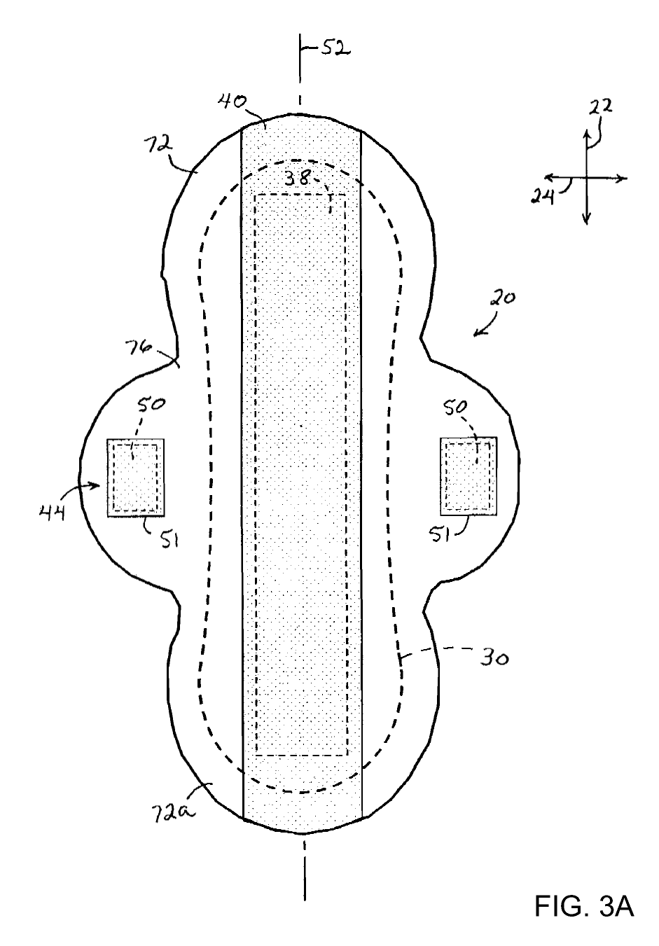

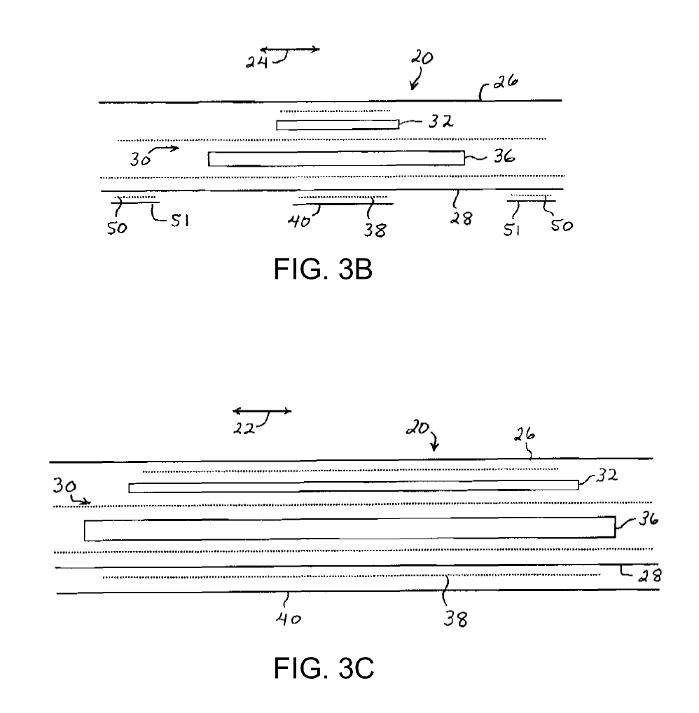

In other configurations, the wings or side-panels 42 can be unitarily formed with one or more components of the article. As representatively shown in FIGS. 2 through 3C, for example, either or both wing portions may be formed from a corresponding, operative extension of the material employed to form the cover 26. Alternatively, either or both wing portions may be formed from a corresponding, operative extension of the material employed to form the baffle 28, or formed from a corresponding, operative combination of the cover and baffle materials.

The side-panels can have an appointed storage position (e.g. FIGS. 1A through 1C) in which the side-panels 42 are directed generally inwardly toward the longitudinally-extending centerline 52. As illustrated, the side-panel that is connected to one side margin may have sufficient cross-directional length to extend and continue past the centerline 52 to approach the laterally opposite side margin of the article. The storage position of the side-panels can ordinarily represent an arrangement observed when article is first removed from its wrapper or other packaging. Prior to placing the article into a bodyside of an undergarment prior to use, the side-panels 42 can be selectively arranged to extend laterally from the side regions of the article intermediate portion (e.g. FIGS. 2 and 2A). After placing the article in the undergarment, the side-panels 42 can be operatively wrapped and secured around the side edges of the undergarment to help hold the article in place.

Additionally, a selected configuration of garment adhesive 38, such as the illustrated strip regions, may be distributed onto the garment-side of the article to help secure the article to the undergarment. Typically, the garment adhesive can be distributed over the garment-side of the baffle, and one or more layers or sheets of release material 40 can be removably placed over the garment adhesive during storage prior to use.

The side-panel portions 42 can have any operative construction, and can include a layer of any operative material. Additionally, each side-panel can comprise a composite material. For example, the side-panels may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web or the like, as well as combinations thereof.

Each side-panel 42 can be joined to its corresponding side region of the article in any operative manner. For example, the side-panel can be joined to the cover 26, the baffle 28 or another article component, as well as any combination thereof. In the illustrated example, each side-panel 42 is joined to the outward, garment-side surface of the baffle 28, but may optionally be joined to the bodyside surface of the baffle. The side-panel can be attached with hotmelt adhesive, but any other operative adhesive or attachment mechanism may alternatively be employed.

In another feature, each side-panel portion 42, or any desired combination of the employed side-panel portions, can include a panel-fastener component 44 which is operatively joined to an appointed engagement surface of its associated side-panel. The panel-fastener can be configured to operatively attach to the wearer's undergarment and/or to any appointed, landing-zone portion of the article 20. For example, the panel-fastener can include a system of interengaging mechanical fasteners, a system of adhesive fasteners, a system of cohesive fasteners or the like, as well as combinations thereof.

With reference to FIGS. 1A through 2C, for example, either or both side-panels 42 can include a hook or other “male” component 46 of an interengaging mechanical fastener system. Any operative hook component may be employed. For example, a suitable hook component materials can include a J-hook, mushroom-head hook, flat-top nail-head hook, a palm-tree hook, a multiple-J hook or the like, as well as combinations thereof.

With reference to FIGS. 3 through 3C, for example, either or both side-panels 42 can include a panel-fastener system 44 which alternatively incorporates an operative adhesive 50. The adhesive may be a solvent-base adhesive, a hotmelt adhesive, a pressure-sensitive adhesive, or the like, as well as combinations thereof. Each section of adhesive 50 may be covered with a removable release sheet 51.

An operative first section of the selected hook component 46 can be joined to a major facing surface of at least a first side-panel portion 42, and can be configured to contact or otherwise engage a cooperating loop material 48 provided on a second side-panel portion 42a during ordinary use, as representatively shown in FIGS. 1A and 1B. Additionally, an operative second section of a hook component 46a, composed of the same or different type of hook material, can be joined to a major facing surface of the second side-panel portion 42a, and can be configured to contact or otherwise engage an outward surface of the wearer's undergarment during ordinary use. For example, the hook component can be arranged to operatively engage and removably attach to the outward surface of a crotch region of the undergarment.

Each side-panel portion 42, or any desired combination of the employed side-panel portions, can include a loop or other “female” component 48 of an interengaging mechanical fastener system. Any operative loop component may be employed. For example, a suitable loop component material can include a woven fabric, a knit fabric, a nonwoven fabric, a fabric laminated to a substrate or the like, as well as combinations thereof. The loop material may be integrally formed with or otherwise provided by the material of its corresponding side-panel portion. Optionally, the loop material may be a separately provided component of that is subsequently assembled to its corresponding side-panel portion.

An operative first section of a selected loop component 48 can be joined to a major facing surface of at least the second side-panel portion 42a, and can be configured to contact or otherwise engage the hook component 46 on the first side-panel portion 42 during ordinary use, as representatively shown in FIGS. 1A and 1B. Additionally, an operative second section of a loop component 48a, composed of the same or different type of loop material, can be joined to a major facing surface of the first side-panel portion 42. As a result, the user can have the option of alternatively attaching the second hook component 46a of the second side-panel onto the second loop component 48a of the first side-panel. Accordingly, the first hook component 46 may alternatively be engaged with the outward surface of the wearer's undergarment.

Each or any desired combination of the provided loop components (48, 48a) may be a separately provided member that is subsequently joined and assembled to its corresponding side-panel portion (42a, 42). In a desired feature, each or any desired combination of the provided loop components can be integrally provided by the material employed to construct its corresponding side-panel portion.

In the various arrangements of the present invention, the hook component 46 can be configured to have a particularly selected hook concentration or density (hooks per unit area). In a particular aspect, the hook density can be at least a minimum of about 1500 hooks/in² (about 232 hooks/cm²). The hook density can alternatively be at least about 2000 hooks/in² (about 310 hooks/cm²), and can optionally be at least about 3000 hooks/in² (about 465 hooks/cm²) to provide improved performance. In another aspect, the hook density can be not more than a maximum of about 7000 hooks/in² (about 1085 hooks/cm²). The hook density can alternatively be not more than about 6000 hooks/in² (about 930 hooks/cm²), and can optionally be not more than about 5000 hooks/in² (about 775 hooks/cm²) to provide improved performance.

Examples of suitable hook materials can include 85-Series and 61-Series hook materials available from Velcro, U.S.A., a business having offices located in Manchester, N.H., U.S.A. The hook materials can have a hook density of about 775 hooks/cm².

In a particular aspect, the material of the loop component 48 may include a nonwoven fabric having continuous bonded areas defining a plurality of discrete unbonded areas. The fibers or filaments within the discrete unbonded areas of the fabric are dimensionally stabilized by the continuous bonded areas that encircle or surround each unbonded area, such that no support or backing layer of film or adhesive is required. The unbonded areas are specifically designed to afford spaces between fibers or filaments within the unbonded area that remain sufficiently open or large to receive and engage hook elements of the complementary hook material. In particular, a pattern-unbonded nonwoven fabric or web may include a spunbond nonwoven web formed of single component or multi-component melt-spun filaments. At least one surface of the nonwoven fabric can include a plurality of discrete, unbonded areas surrounded or encircled by continuous bonded areas. The continuous bonded areas dimensionally stabilize the fibers or filaments forming the nonwoven web by bonding or fusing together the portions of the fibers or filaments that extend outside of the unbonded areas into the bonded areas, while leaving the fibers or filaments within the unbonded areas substantially free of bonding or fusing. The degree of bonding or fusing within the bonding areas desirably is sufficient to render the nonwoven web non-fibrous within the bonded areas, leaving the fibers or filaments within the unbonded areas to act as “loops” for receiving and engaging hook elements. Examples of suitable point-unbonded fabrics are described in U.S. Pat. No. 5,858,515, the entire disclosure of which is incorporated herein by reference in a manner that is consistent herewith.

The formulations of the present disclosure may be applied to the absorbent article in any suitable location including, for example, on the cover, the wings, the intake layer, the absorbent shaping layer, combinations of these locations, and the like. In one particular embodiment, the formulation may be applied to the absorbent article along the perimeter of the article. In this embodiment, the formulation will preferably comprise an anti-adherence agent. By applying the formulation along the perimeter of the absorbent article, the formulation (and in particular the anti-adherence agent present in the formulation) acts as a barrier, preventing exudates from passing out the side of the article, thus reducing side leakage.

In another embodiment, the formulation is coated on a skin contacting surface of the absorbent article, such as on the body-facing side of the cover and/or wings of the absorbent article. In this manner, the formulation may be transferred from the absorbent article to the skin of the article wearer to help improve skin health and cleanliness. In one particular embodiment, the formulation applied to the absorbent article may comprise an anti-adherence agent and/or a viscoelastic agent. Once the formulation is transferred to the body of the wearer, the presence of the anti-adherence agent on the body of the wearer may help prevent exudates from sticking to the body, while the viscoelastic agent may help promote transport of the exudate off the body and into the absorbent article. Additionally, the presence of the viscoelastic agent on the absorbent article may further reduce the viscoelastic properties of the exudate, thus promoting more complete absorption into the absorbent article.

In another embodiment, the formulation may first be applied to the skin using a wipe impregnated with a formulation of the present invention prior to or after donning of the absorbent article. In this manner, additional formulation may be transferred to the skin to further improve skin health and cleanliness.

Thus in another aspect, the present disclosure is directed to a system for skin health and cleanliness. The system may comprise a formulation of the present disclosure, an absorbent article comprising a formulation of the present disclosure, a wipe having a formulation of the present disclosure impregnated therein, or some combination thereof. The formulation, wipe, and/or absorbent article may be used in combination to improve the moisturization, smoothness, and elasticity of skin, while simultaneously preventing exudates from sticking to the body and promoting transport of exudates away from the body and towards the absorbent article.

In one particular embodiment, the system may comprise a formulation of the present disclosure and/or a wipe having a formulation of the present disclosure impregnated therein. The system may further comprise an absorbent article, as described herein, which may be used in combination with the formulation and/or wipe. As noted above, the absorbent article may have a formulation of the present disclosure incorporated therein. Alternately, instead of the formulation, the absorbent article may comprise a viscoelastic agent and/or an anti-adherence agent to help improve the ability of the absorbent article to absorb exudates coming into contact with the article.

Having described the disclosure in detail, it will be apparent that modifications and variations are possible without departing from the scope of the disclosure defined in the appended claims.

### DEFINITIONS

As used herein the term “viscoelastic” means a composition having at least one significant component that is moderately viscous and has elastic properties. By “moderately viscous” it is meant that the component has a viscosity of at least that of normal human blood plasma. By “elastic” it is meant that the component has elasticity greater than normal human blood plasma.

The term “viscoelastant” or “viscoelastic agent,” used interchangeably herein, means an organic agent that, when an effective amount is contacted by a viscoelastic composition, materially alters the properties of that viscoelastic composition, for example, by reducing its viscosity and/or elastic nature. By “materially alters” it is meant that the property measured as described is changed by at least a statistically significant amount and, advantageously, this change will be at least about 30% for many applications.

As used herein the term “nonwoven fabric or web” means a web having a structure of individual fibers or threads which are interlaid, but not in a regular or identifiable manner as in a knitted fabric. The term also includes individual filaments and strands, yarns or tows as well as foams and films that have been fibrillated, apertured, or otherwise treated to impart fabric-like properties. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein the term “microfibers” means small diameter fibers having an average diameter not greater than about 75 microns, for example, having an average diameter of from about 0.5 microns to about 50 microns, or more particularly, microfibers may have an average diameter of from about 2 microns to about 40 microns. Another frequently used expression of fiber diameter is denier, which is defined as grams per 9000 meters of a fiber and may be calculated as fiber diameter in microns squared, multiplied by the density in grams/cc, multiplied by 0.00707. A lower denier indicates a finer fiber and a higher denier indicates a thicker or heavier fiber. For example, the diameter of a polypropylene fiber given as 15 microns may be converted to denier by squaring, multiplying the result by 0.89 g/cc and multiplying by 0.00707. Thus, a 15 micron polypropylene fiber has a denier of about 1.42 (152×0.89×0.00707=1.415). Outside the United States the unit of measurement is more commonly the “tex”, which is defined as the grams per kilometer of fiber. Tex may be calculated as denier/9.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as, for example, described in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Levy, and U.S. Pat. No. 3,542,615 to Dobo et al. Spunbond fibers are quenched and generally not tacky when they are deposited onto a collecting surface. Spunbond fibers are generally continuous and have average diameters frequently larger than 7 microns, more particularly, between about 10 and 20 microns.

As used herein the term “meltblown fibers” means fibers formed by extruding a molten thermoplastic material through a plurality of fine, usually circular, die capillaries as molten threads or filaments into converging high velocity, usually heated, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface often while still tacky to form a web of randomly dispersed meltblown fibers. Such a process is disclosed, for example, in U.S. Pat. No. 3,849,241 to Butin. Meltblown fibers are microfibers which may be continuous or discontinuous and are generally smaller than 10 microns in average diameter.

As used herein “bonded carded webs” or “BCW” refers to nonwoven webs formed by carding processes as are known to those skilled in the art and further described, for example, in coassigned U.S. Pat. No. 4,488,928 to Alikhan and Schmidt which is incorporated herein in its entirety by reference. Briefly, carding processes involve starting with a blend of, for example, staple fibers with bonding fibers or other bonding components in a bulky batt that is combed or otherwise treated to provide a generally uniform basis weight. This web is heated or otherwise treated to activate the adhesive component resulting in an integrated, usually lofty nonwoven material.

As used here, “airlaid” refers to nonwovens formed by airlaying processes. “Airlaying” is a well-known process by which a fibrous nonwoven layer can be formed. In the airlaying process, bundles of small fibers having typical lengths ranging from about 3 to about 19 millimeters (mm) are separated and entrained in an air supply and then deposited onto a forming screen, usually with the assistance of a vacuum supply. The randomly deposited fibers then are bonded to one another using, for example, hot air or a spray adhesive. Airlaying is taught in, for example, U.S. Pat. No. 4,640,810 to Laursen et al. As used herein, “coform” is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configuration of the material. These configurations include, but are not limited to isotactic, syndiotactic and random symmetries.

As used herein the term “blend” as applied to polymers, means a mixture of two or more polymers.

As used herein, through air bonding or “TAB” means a process of bonding a nonwoven, for example, a bicomponent fiber web in which air which is sufficiently hot to melt one of the polymers of which the fibers of the web are made is forced through the web. The air velocity is often between 100 and 500 feet per minute and the dwell time may be as long as 6 seconds. The melting and resolidification of the polymer provide the bonding. Through air bonding has restricted variability and is often regarded a second step bonding process. Since TAB requires the melting of at least one component to accomplish bonding, it is restricted to webs with two components such as bicomponent fiber webs or webs containing an adhesive fiber, powder or the like. TAB is frequently used to bond BCW materials.

As used herein “thermal point bonding” involves passing a fabric or web of fibers to be bonded between a heated calender roll and an anvil roll. The calender roll is usually, though not always, patterned in some way so that the entire fabric is not bonded across its entire surface. As a result, various patterns for calender rolls have been developed for functional as well as aesthetic reasons. One example of a pattern has points and is the Hansen Pennings or “H&P” pattern with about a 30% bond area with about 200 bonds/square inch as taught in U.S. Pat. No. 3,855,046 to Hansen and Pennings. The H&P pattern has square point or pin bonding areas wherein each pin has a side dimension of 0.038 inches (0.965 mm), a spacing of 0.070 inches (1.778 mm) between pins, and a depth of bonding of 0.023 inches (0.584 mm). The resulting pattern has a bonded area of about 29.5%. Another typical point bonding pattern is the expanded Hansen and Pennings or “EHP” bond pattern which produces a 15% bond area with a square pin having a side dimension of 0.037 inches (0.94 mm), a pin spacing of 0.097 inches (2.464 mm) and a depth of 0.039 inches (0.991 mm). Another typical point bonding pattern designated “714” has square pin bonding areas wherein each pin has a side dimension of 0.023 inches, a spacing of 0.062 inches (1.575 mm) between pins, and a depth of bonding of 0.033 inches (0.838 mm). The resulting pattern has a bonded area of about 15%. Yet another common pattern is the C-Star pattern which has a bond area of about 16.9%. The C-Star pattern has a cross-directional bar or “corduroy” design interrupted by shooting stars. Other common patterns include a diamond pattern with repeating and slightly offset diamonds and a wire weave pattern looking as the name suggests, e.g. like a window screen. Typically, the percent bonding area varies from around 10% to around 30% of the area of the fabric laminate web. As in well known in the art, the spot bonding holds the laminate layers together as well as imparts integrity to each individual layer by bonding filaments and/or fibers within each layer.

### EXAMPLES

The following non-limiting examples are provided to further illustrate the present disclosure.

### Example 1

In this example, a lotion formulation comprising a coefficient of friction (COF) modulator, a moisturizer, a skin texture modulator, an elasticity modulator, an anti-adherence agent, and a temperature modulator was prepared. The following components were used to prepare the formulation.

TABLE 1

| | | | |
|---|---|---|---|
| Trade Name | INCI Name | Weight % | Function |

Phase 1

| | | | |
|---|---|---|---|
| Water | Water | 56 | Aqueous carrier |
| VERSENE Na₂ | Disodium EDTA | 0.1 | Chelating agent |
| (Dow) | | | |

Phase 2

| | | | |
|---|---|---|---|
| Glycerin | Glycerin | 9 | Moisturizer |
| Inulin | Inulin | 0.1 | Anti-adherence agent |

Phase 3

| | | | |
|---|---|---|---|
| DC 200 100CST | Dimethicone | 5 | COF modulator |
| fluid | | | Temperature modulator |
| | | | Emollient |
| Vitamin E | Tocopheryl acetate | 3 | Elasticity modulator |
| acetate | | | |
| Mineral Oil | Mineral Oil | 10 | COF modulator |
| | | | Temperature modulator |
| | | | Emollient |
| ABIL CARE 85 | Bis-PEG/PPG-16/16; | 2 | Emulsifier |
| | PEG/PPG-16/16; | | |
| | dimethicone; caprylic/capric | | |
| | triglyceride | | |
| CERAPHYL SLK | Isodecyl neopentanoate | 3 | Skin conditioner |

Phase 4

| | | | |
|---|---|---|---|
| Nylon-12 | Nylon-12 | 5 | COF modulator |
| | | | Skin texture modulator |

Phase 5

| | | | |
|---|---|---|---|
| RM2051 (Dow | Sodium polyacrylate (and) | 6 | Emulsifier/rheology modifier |
| Corning) | dimethicone (and) | | |
| | cyclopentasiloxane (and) | | |
| | trideceth-6 (and) | | |
| | PEG/PPG-18/18 | | |
| | dimethicone | | |

Phase 6

| | | | |
|---|---|---|---|
| PARAGON | Phenoxyethanol; | 0.8 | Preservative |
| MEPB | methylparaben; | | |
| | ethylparaben; | | |
| | propylparaben; | | |
| | butylparaben; | | |
| | isobutylparaben | | |

Phase 7

| | | | |
|---|---|---|---|
| KOH 10% | Potassium hydroxide | q.s. | pH adjuster |
| solution by | | | |
| weight of the | | | |
| solution | | | |

q.s. = quantum satis

The composition was prepared using the following procedure:

1) The Phase 1 materials were combined, and mixing was begun.

2) The Phase 2 materials were premixed until dispersed, and then added to the Phase 1 materials with moderate to high mixing.

3) The Phase 3 materials were premixed until substantially homogenous, and then added to the Phase 1 and 2 mixture with moderate to high mixing.

4) Phase 4 (i.e., Nylon-12) was added to the mixture and mixed until dispersed.

5) Phase 5 (i.e., RM2051) was added to the mixture with moderate to high mixing.

6) Phase 6 (i.e., the preservatives) was added and mixed until the resulting mixture was substantially homogenous.

7) The KOH was added to Q.S. until the pH of the mixture was about 6.0.

### Example 2

In this example, lotion formulations comprising varying amounts of a coefficient of friction modulator, moisturizer, skin texture modulator, and elasticity modulator were prepared. The lotions were prepared as described in Example 1, using the same components and amounts with the following exceptions.

For each formulation, the moisturizer, coefficient of friction modulator, skin texture modulator, and elasticity modulator were included in the formulation in either a high or low amount. In particular, the moisturizer, glycerin, was included in an amount of either 3 wt. % (low) or 9 wt. % (high); the coefficient of friction modulator, dimethicone, was included in an amount of either 1 wt. % (low) or 5 wt. % (high); the skin texture modulator, Nylon-12, was included in an amount of either 1 wt. % (low) or 5 wt. % (high); and the skin elasticity modulator, Vitamin E acetate, was included in the composition in an amount of either 0.5 wt. % (low) or 3 wt. % (high). Sixteen different formulations were created by varying the amounts of these four components. The specific amounts of these components present in each formulation are set forth in Table 2 below. The total amount of water included in each formulation was adjusted accordingly to account for the varying amounts of glycerin, dimethicone, Nylon-12, and Vitamin E acetate.

TABLE 2

| | | | | Vitamin E |
|---|---|---|---|---|
| | Glycerin | Dimethicone | Nylon-12 | acetate |
| Lotion | (wt. %) | (wt. %) | (wt. %) | (wt. %) |
| 1 | 3% | 1% | 1% | 0.5% |
| 2 | 9% | 1% | 1% | 0.5% |
| 3 | 3% | 5% | 1% | 0.5% |
| 4 | 9% | 5% | 1% | 0.5% |
| 5 | 3% | 1% | 5% | 0.5% |
| 6 | 9% | 1% | 5% | 0.5% |
| 7 | 3% | 5% | 5% | 0.5% |
| 8 | 9% | 5% | 5% | 0.5% |
| 9 | 3% | 1% | 1% | 3% |
| 10 | 9% | 1% | 1% | 3% |
| 11 | 3% | 5% | 1% | 3% |
| 12 | 9% | 5% | 1% | 3% |
| 13 | 3% | 1% | 5% | 3% |
| 14 | 9% | 1% | 5% | 3% |
| 15 | 3% | 5% | 5% | 3% |
| 16 | 9% | 5% | 5% | 3% |

### Example 3

Static coefficient of friction values can be used as an indication of shear forces that occur between the skin and materials that may contact the skin. In particular, lower static coefficient of friction values indicate lower shear forces between the skin and the material, and increased gentleness. In this example, the lotion formulations prepared as described in Example 2 were tested to evaluate the effect of each formulation on skin coefficient of friction as tested against three different substrates.

The substrates tested were silk habutae, a non-woven material (i.e., 20 grams per square meter (gsm) spunbond (BBA Fiberweb, Simpsonville, S.C.)), and hydrated Vitro Skin® N19-5X (IMS, Milford, Conn.). Prior to testing, the Vitro-Skin® substrate was hydrated for 20 hours in a hydration chamber in a controlled environment at 23° C. and 50% relative humidity.

Each substrate was cut into 3×5 cm pieces and allowed to equilibrate in a controlled environment at 23° C. and 50% relative humidity for at least thirty minutes before use.

A Monitor/slip and friction instrument (TMI, Amityville, N.Y., model 32-06) was used to conduct friction measurements. All tests were conducted in a controlled environment (23° C. and 50% relative humidity). A modified sled was made to have a bottom surface area of 5 cm×3 cm and a weight of 73 g. The Monitor/slip and friction instrument was set to move the sled at a rate of 25.4 cm/min, and to stop after traveling 7.00 cm. A 700 g weight was placed on the sled before testing began for extra weight on the substrate.

The measurements from the Monitor/slip friction instrument were compiled with a custom software program (Pau-Lin Pawar, AMT, Neenah, Wis.). The normal force was set at 774 g to account for the sled, the 700 g weight, the test samples, and clips. The forward velocity of the sled was 10 in/min.

All tests were performed in an environmentally controlled room (23° C. and 50% relative humidity). The test substrate (either the silk habutae, non-woven material, or hydrated Vitro Skin® N19-5X) was clipped to the lip of the sled using three mini binder clips (EXP, Broomfield, Colo.), one at the top and one on each side. The finger grips of the clips were bent up so they did not touch the skin simulant during testing. A piece of Vitro Skin® N19-5X was placed on the shiny side of a 5 cm×15 cm piece of silicon skin (SiliClone, Valley Forge, Pa.). Using a positive displacement pipette, 200 μL of the lotion formulation being tested was applied down the center of the Vitro Skin® substrate and rubbed in with a gloved finger. The silicone skin and edge of the treated Vitro Skin® substrate were secured to the instrument with the Monitor/slip and friction instrument sample retaining clip. The sled, with the test substrate attached, was placed into position on the instrument and lined up over the treated Vitro Skin® substrate. The experiment was carried out by pressing the start button for the software then the test button on the instrument. A new piece of Vitro Skin® substrate and test substrate was used for each test, and one wipe was conducted per test. A control test was also run five times for each test substrate without the application of lotion to the Vitro Skin® substrate.

The lotion formulations were evaluated by comparing the static friction, or the force required to start the test substrate moving. The static friction value was recorded as the force measurement that corresponded to the top of the initial peak from the force curve measured in grams force (gf). Each lotion formulation was tested five times for each test substrate, and the static coefficient of friction for the tests for each substrate was averaged. Differences between static coefficient of friction values were determined by using analysis of variance between groups.

Results

Application of every lotion formulation to the Vitro Skin® substrate reduced the frictional force value between the treated Vitro Skin® substrate and the silk and the nonwoven spunbond test substrates. Application of most lotion formulations to the Vitro Skin® substrate also reduced the frictional force between the treated Vitro Skin® substrate and the Vitro Skin® test substrate. Three lotion formulations, i.e., lotions 5, 9, and 16, showed significant differences from most of the others on all three test substrates. The maximum force values for lotions 16 and 5 were consistently high while the values for lotion 9 were consistently low relative to the other lotions.

The mean force values (n=5) for each lotion formulation as tested against the three test substrates are given in Table 3 below.

TABLE 3

| | Mean Force Values (gf) | | |
|---|---|---|---|
| Lotion | Silk | Nonwoven | Vitro Skin ® |
| 1 | 326.65 | 276.56 | 200.40 |
| 2 | 338.08 | 311.02 | 198.78 |
| 3 | 329.62 | 303.82 | 189.30 |
| 4 | 345.60 | 328.32 | 194.66 |
| 5 | 355.36 | 331.50 | 246.74 |
| 6 | 346.12 | 296.86 | 208.26 |
| 7 | 367.64 | 301.04 | 206.86 |
| 8 | 335.30 | 312.76 | 244.80 |
| 9 | 305.08 | 273.72 | 170.70 |
| 10 | 327.96 | 280.98 | 198.24 |
| 11 | 341.00 | 289.62 | 191.90 |
| 12 | 346.56 | 284.04 | 168.34 |
| 13 | 348.54 | 296.86 | 228.06 |
| 14 | 317.56 | 324.34 | 244.16 |
| 15 | 338.40 | 282.98 | n/a |
| 16 | 387.08 | 333.46 | 238.97 |
| Control | 532.7 | 426.1 | 229.1 |
| (Untreated) | | | |

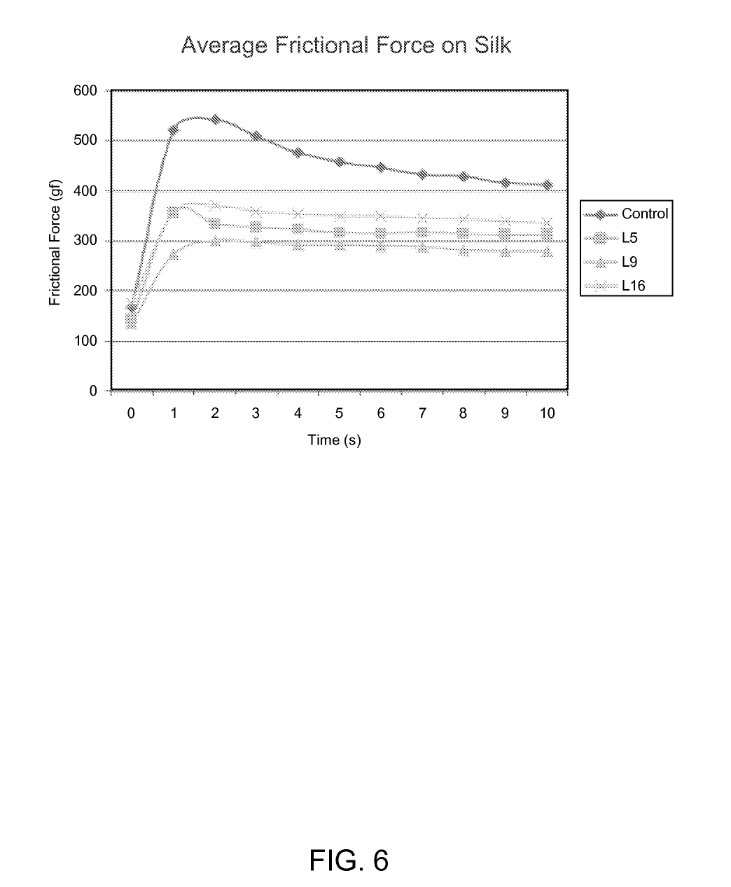

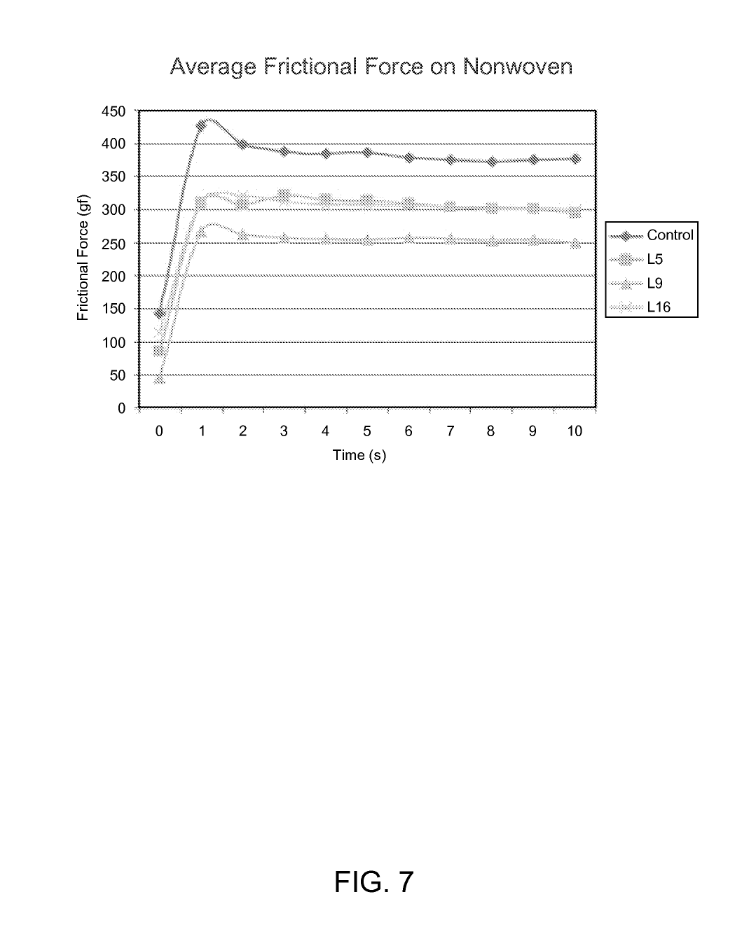

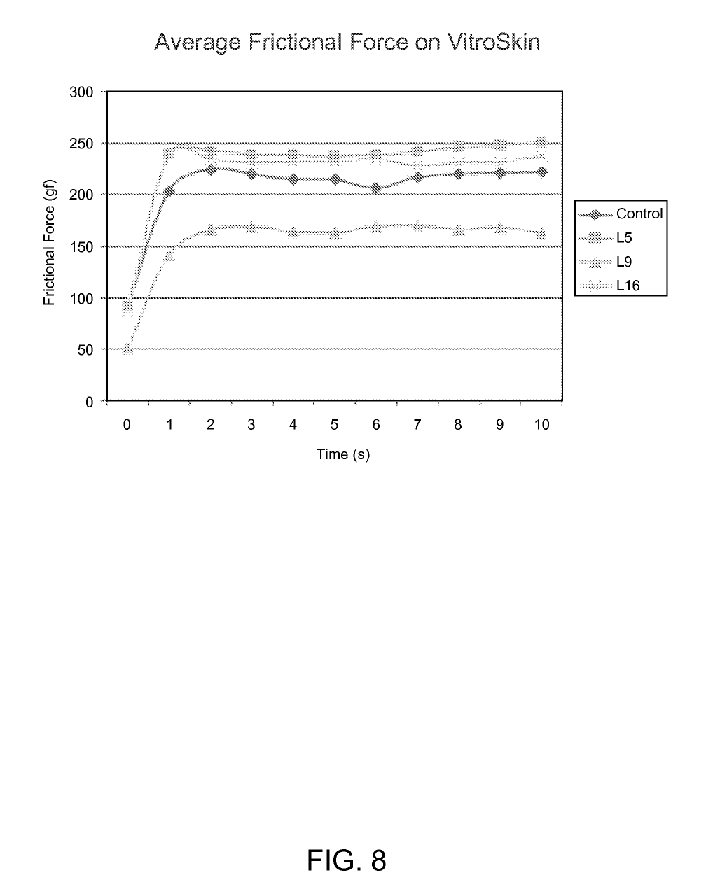

Force curves were generated by plotting frictional force (gf) against time in seconds. The force value at each time point was averaged for each sample (n=5). The results for lotions 5, 9, and 16 are shown in FIGS. 6-8. The formulation used to create lotion 9 reduced the frictional force between the Vitro-Skin® substrate and all three test substrates the most.

### Example 4

The sixteen lotions prepared in Example 2 were evaluated for their effect on skin moisturization, skin temperature, skin texture, and skin elasticity.

Thirty-six female subjects between the ages of 18 and 50 were recruited for each study. Individuals with abnormal skin pigmentation at the test sites, skin disease, skin damage, skin damage due to sun exposure, tattoos or bruises on the testing areas of the arms, or excessive dryness or erythema were excluded. The subjects were instructed not to use skin creams, oils, ointment, powders, perfumes, or lotions on the arms less than 24 hours prior to and during testing. Each subject tested two lotions on each arm and one untreated control site on one arm. Subjects came in for two visits approximately four hours apart.

On the day of testing, the subjects were acclimated to a temperature and humidity controlled room (70°±2° F.; 40%±5% relative humidity) for 15 minutes prior to baseline testing. During equilibration, three 3 cm×3 cm sites were demarcated on the volar aspect of each forearm. After equilibration, baseline measurements were taken for skin temperature, skin texture (roughness), skin moisture (conductance), and skin elasticity.

Skin temperature measurements were taken at each test site using a MiniTemp™ infrared thermometer (Raytek® Corporation, Santa Cruz, Calif.). The thermometer head was held about one inch above the skin surface and the measurement was taken.

Following the temperature measurements, baseline skin texture (roughness) measurements were taken using a Surveyor 3D Laser Profilometer (Laser Design Inc., Minneapolis, Minn.). The arm was rested in a cradle and a two minute laser scan of the test site was taken. Roughness calculations were performed using TalyMap Universal® v3.1.10 software. The topographic parameter Sₐ (mean absolute deviation) was used to calculate roughness.

Following roughness measurements, baseline conductance measurements were taken at each site using a DermaLab Moisture Flat Probe (Cortex Technology, Hadsund Denmark). Conductance is the cosmetic industry standard for measuring moisture in the skin. The DermaLab Moisture Flat Probe was used for all measurements. It uses electrodes arranged as concentric rings to send a series of alternating electrical currents through the skin. Resistance to the currents indicates the water binding capacity of the stratum corneum, or moisture level, and provides a conductance reading. A higher conductance reading indicates a higher level of moisture in the skin. The instrument's probe was placed at the test site and 5 second continuous measurements were taken in triplicate.

Following conductance measurements, baseline elasticity measurements were taken at each site using a DermaLab Elasticity Probe (Cortex Technology, Hadsund, Denmark). The probe was sealed well against the skin using an adhesive disc, and one measurement of 5 cycles was taken at each test site. During each cycle, a small area of the skin was pulled by suction to two elevation detectors, one positioned at 1 mm above the skin and the other at 2.5 mm above the skin. The elasticity measurement was an average of the difference between the pressures required to pull the skin up these amounts. A lower difference indicates more hydrated, supple skin, and increased elasticity and moisturization.

At the conclusion of baseline measurements, 2 mg/cm² (18 μL) of the lotion formulation being tested was applied with a positive displacement pipette to a test site. Each test site received a different lotion formulation according to a randomization schedule. The lotion formulations were rubbed into the test site for approximately 10 seconds using a gloved finger. One test site on each subject served as the untreated control and received no lotion, but was rubbed for approximately 10 seconds to simulate lotion application.

Subjects were allowed to leave test site after application of the lotion formulations, and returned about fifteen minutes prior to the final measurements. The subjects were reacclimated to the temperature and humidity controlled environment for at least fifteen minutes, as described above. Final measurements of skin temperature, roughness, conductance, and elasticity were made 4 hours after the baseline measurements were taken, using the procedures described above.

Skin Temperature Measurements

Skin temperature varies for different regions of the body but typically remains between about 32° C. (90° F.) and about 35° C. (95° F.). In order to be comfortable, a skin temperature of 33° C. (91° F.) should be maintained. Preferred lotion formulations will allow for maintaining skin temperature at normal temperatures after application for up to 4 hours.

All of the lotion formulations tested maintained skin temperature at normal temperatures after application for up to 4 hours. Skin temperature measurements ranged from 87° F. to 95° F. The largest difference between a baseline reading and a final reading was 3° F.

Moisture Measurements

As noted above, the conductance measurements for each formulation were taken in triplicate. The conductance measurements for each formulation were averaged, and the difference between the test measurements at four hours and the baseline measurements was calculated. The average value for the untreated sites was then subtracted from these differences and the results were compared using analysis of variance.

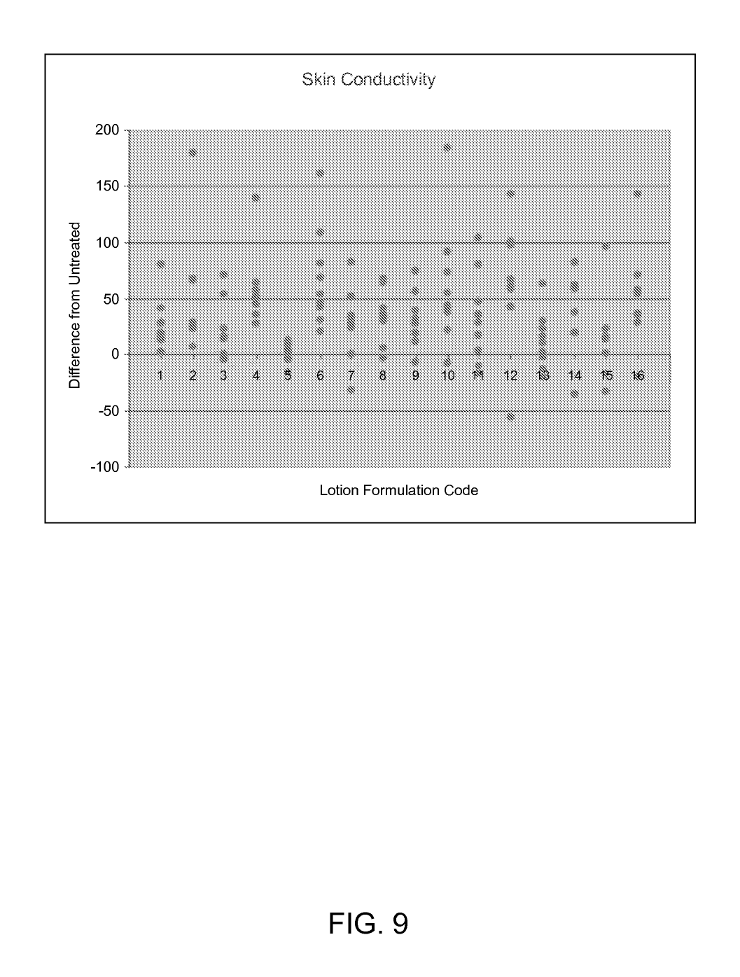

Preferably, the lotion formulations will increase stratum corneum moisturization significantly more than no treatment up to and including 4 hours after application. The results are shown in FIG. 9, which illustrates the difference in actual conductance measurements for skin treated with each lotion as compared to the untreated skin. Positive numbers indicate an increase in conductance relative to untreated skin. The lotion formulations that increased conductance the most (and thus resulted in the greatest increase in moisturization) were lotions 2, 4, 6, 8, 10, 12, 14, and 16. In all of these formulations, the moisturizer, glycerin, was present at the high level. Lotions 6, 12, 10, and 4 showed the greatest, consistent increase in conductivity as compared to the untreated values, and were significantly different from the lotions that showed the least increase in conductance.

Skin Texture Measurements

The difference between the roughness value at four hours for each lotion and its baseline was calculated, and the average of the measurements for the untreated sites was subtracted from these quantities. These resulting values were compared using analysis of variance.

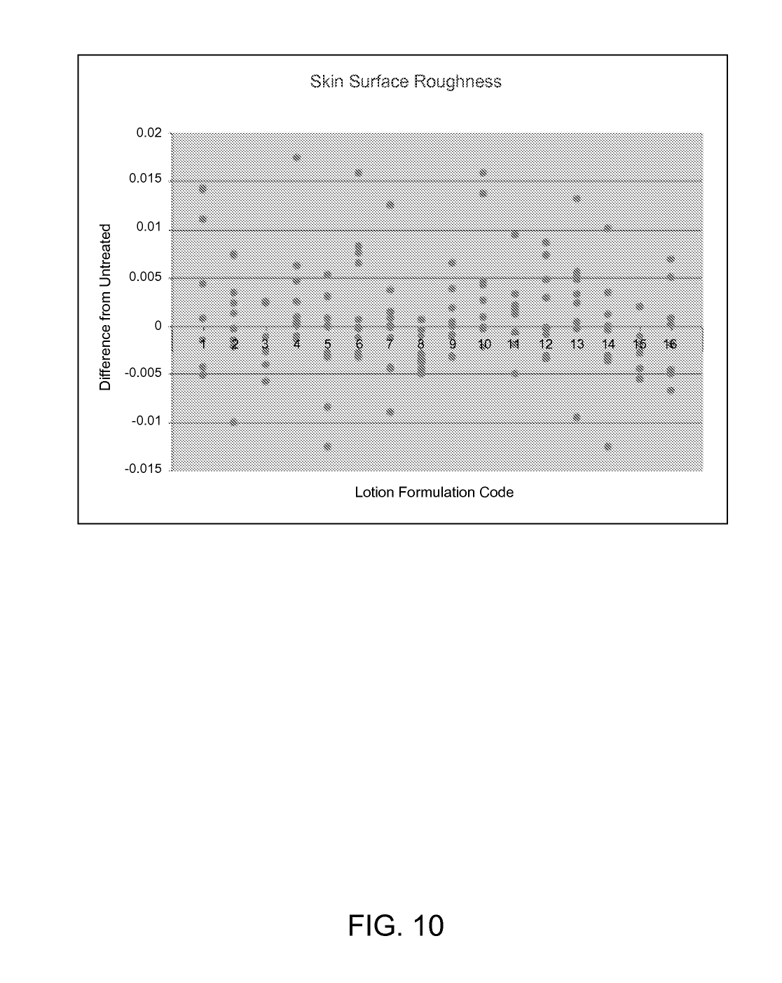

The results are shown in FIG. 10, which illustrates the difference in actual roughness value for skin treated with each lotion as compared to the untreated skin. Negative numbers indicate smoother skin (i.e., a decrease in roughness), while positive numbers indicate rougher skin (i.e., an increase in roughness) as compared to the untreated skin. The lotions that decreased roughness were lotions 3, 8, 14, 15, and 16. Four out of the five lotions that decreased roughness contained the high level of Nylon-12, a skin texture modulator. Lotion 8 decreased skin surface roughness the most, and based on statistical analysis was significantly different from the lotions that caused an increase in roughness.

Skin Elasticity Measurements

The difference between the elasticity value for each lotion tested and its baseline was calculated and the average measurement for the untreated sites was subtracted from these values. The resulting values were compared using analysis of variance. The formulations that increased elasticity were lotions 3, 9, 12, 15, and 16. Four out of the five lotions that increased elasticity contained the high level of the elasticity modulator, Vitamin E acetate. Lotions 3 and 9 increased skin elasticity the most, and based on statistical analysis, were significantly different from the lotions that caused a decrease in elasticity.

### Example 5

In this example, the ability of a dimethicone-containing formulation to affect exudate adherence to skin was evaluated.

A commercially available dimethicone-based formulation was tested. The following components were present in the formulation.

TABLE 4
INCI Name

Dimethicone
Cyclopentasiloxane
Dimethicone/vinyl dimethicone crosspolymer
Silica
Tocopheryl acetate
Trisiloxane

All materials used in this example, including KOTEX® Ultra Thin Maxi pads (available from Kimberly-Clark Corporation), the dimethicone-based gel described above, and VITRO-SKIN® synthetic skin substrate, were acclimated for 1 hour in a heated, humidified chamber at 30° C. and 80% relative humidity.

The VITRO-SKIN® substrate was cut into 10 cm×10 cm squares, and the mass of the squares of VITRO-SKIN® substrate and the mass of the pads was measured. 0.1 g of the dimethicone-based gel was spread evenly onto a 5 cm×5 cm area of the VITRO-SKIN® substrate using an index finger, and the substrate was allowed to stand for 1 minute. 0.5 g of a menses simulant was dispensed onto the treated VITRO-SKIN® substrate, spread over a 3 cm×3 cm area with an index finger, and allowed to stand for 1 minute. The pad was positioned over the VITRO-SKIN® substrate so that the middle of the pad contacted the menses simulant. A 1.6 kg (5×8 cm) weight was placed on top of the pad. The weight was removed from the pad after 1 minute, and the mass of the pad and the VITRO-SKIN® substrate was measured, to determine the degree of transfer of menses simulant from the VITRO-SKIN® substrate to the pad. A control was also run three times using a VITRO-SKIN® substrate treated with menses simulant, but no dimethicone-based gel.

For the control, the average for the three control tests showed that 18% of the menses simulant remained on the untreated VITRO-SKIN® substrate after contact with the pad. In contrast, 0% of the menses simulant remained on the VITRO-SKIN® substrate that was treated with the dimethicone-based gel. These results indicate dimethicone-based gel may enhance the removal of menses from skin.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above compositions and products without departing from the scope of the disclosure, it is intended that all matter contained in the above description shall be interpreted as illustrative and not in a limiting sense.

What is claimed is:

1. A formulation for promoting skin cleanliness and health, the formulation comprising from about 0.01% (by total weight of the formulation) to about 10% (by total weight of the formulation) of a coefficient of friction modulator; from about 1% (by total weight of the formulation) to about 15% (by total weight of the formulation) of a moisturizer; from about 0.01% (by total weight of the formulation) to about 10% (by total weight of the formulation) of an elasticity modulator; from about 0.01% (by total weight of the formulation) to about 10% (by total weight of the formulation) of a skin texture modulator; and from about 1% (by total weight of the formulation) to about 99% (by total weight of the formulation) of a pharmaceutically acceptable carrier.

2. The formulation of claim 1 wherein the coefficient of friction modulator is selected from the group consisting of polydimethylsiloxane, dimethicone, silicone crosspolymers, silicone gums, dimethiconol, trimethylsilyl, amodimethicone, silsesquioxanes, alkylmethylsiloxanes, silicone wax, silanes, silica, fumed silica, propylene glycol, 1,2-propanediol, stearalkonium chloride, starch, cyclomethicone, mineral oil, petrolatum, polymethacrylate, polymethylmethacylate, aluminum starch octenylsuccinate, calcium starch octenylsuccinate, blends of aluminum starch octenylsuccinate and lauroyl lysine, blends of aluminum starch octenylsuccinate and boron nitride, Nylon-12, Nylon-6, nanoparticles, carboxymethylcellulose, ethyl cellulose, hydroxyethylcellulose, silicone polymers, molybdenum disulfide, polytetrafluoroethylene, insoluble starch, lipids, cross-linked alginate, dextrin, graphite, cross-linked vinylpyrrolidone homopolymers, chitin, polypropylene, pectin, talc, cationic polymers, and combinations thereof.

3. The formulation of claim 1 wherein the formulation comprises the coefficient of friction modulator in an amount of from about 0.1% (by total weight of the formulation) to about 8% (by total weight of the formulation).

4. The formulation of claim 1 wherein the moisturizer is selected from the group consisting of glycerin, ethoxylated glycerin, sodium lactate, lactic acid, glycolic acid, urea, hydrolyzed proteins, hyaluronic acid, salicylic acid, phospholipids, propylene glycol, butylene glycol, caprylyl glycol, ethoxylated propylene glycol, glycerol, collagen, pyrrolidone carboxylic acid PCA, sodium PCA, betaine, diglycerin, glucose, sucrose, xylitol, fructose, sorbitol, mannitol, hydrolyzed starch, and combinations thereof.

5. The formulation of claim 1 wherein the formulation comprises the moisturizer in an amount of from about 2% (by total weight of the formulation) to about 10% (by total weight of the formulation).

6. The formulation of claim 1 wherein the elasticity modulator is selected from the group consisting of β-glucan, glycerin, lactic acid, glycolic acid, urea, hydrolyzed proteins, dimethylaminoethanol, α-hydroxy acids, liposome collagen, collagen, Vitamin C, Vitamin E, Vitamin A, vitamin derivatives, elastin, peptides, and combinations thereof.

7. The formulation of claim 1 wherein the formulation comprises the elasticity modulator in an amount of from about 0.1% (by total weight of the formulation) to about 8% (by total weight of the formulation).

8. The formulation of claim 1 wherein the skin texture modulator is selected from the group consisting of acrylics, acrylics multipolymer, cellulose nitrate, polypropylene (unmodified), polybutylene, ionomers, polyethylene (low density), polyethylene (medium density), Nylon-6, Nylon-12, styrene butadiene thermoplastic copolymer, polyvinylchloride (PVC) (rigid), polymethylmethacylate, aluminum starch octenylsuccinate, lauroyl lysine, calcium starch octenylsuccinate, boron nitride, polymethacrylate, nanoparticles, carboxymethylcellulose, micronized silicone polymers, graphite, cross-linked vinylpyrrolidone homopolymers, silica, ethylcellulose resins, micronized chitin, micronized polypropylene, pectin, hydroxyethylcellulose, talc, cationic polymers, silsesquioxanes, starch, polymethacrylate, cationics, stearalkonium chloride, glycols, and combinations thereof.

9. The formulation of claim 1 wherein the formulation comprises the skin texture modulator in an amount of from about 0.1% (by total weight of the formulation) to about 8% (by total weight of the formulation).

10. The formulation of claim 1 further comprising a temperature modulator.

11. The formulation of claim 10 wherein the temperature modulator is selected from the group consisting of insulating agents, warming agents, cooling agents, and combinations thereof.

12. The formulation of claim 10 wherein the formulation comprises the temperature modulator in an amount of from about 0.1% (by total weight of the formulation) to about 15% (by total weight of the formulation).

13. The formulation of claim 1 wherein the formulation further comprises an anti-adherence agent.

14. The formulation of claim 13 wherein the anti-adherence agent is selected from the group consisting of alginic acid, β-benzal-butyric acid, botanicals, casein, dextrans, farnesol, flavones, fucans, galactolipid, high molecular weight kininogen, hyaluronate, inulin, iridoid glycosides, nanoparticles, perlecan, phosphorothioate oligodeoxynucleotides, pluronic surfactants Poloxamer 407, polymethylmethacrylate, pluronic surfactants silicone, sulphated exopolysaccharides, tetrachlorodecaoxide, inulin, polymethylmethacrylate, methyl methacrylate crosspolymer, ethylene/acrylate copolymer, polymethylsilsesquioxane, silicone resins, dimethicone and related compounds, and combinations thereof.

15. The formulation of claim 13 wherein the formulation comprises the anti-adherence agent in an amount of from about 0.01% (by total weight of the formulation) to about 5% (by total weight of the formulation).

16. The formulation of claim 13 wherein the coefficient of friction modulator is dimethicone, the moisturizer is glycerin, the elasticity modulator is vitamin E acetate, the skin texture modulator is Nylon-12, and the anti-adherence agent is inulin.

17. The formulation of claim 1 wherein the formulation further comprises a viscoelastic agent.

18. The formulation of claim 17 wherein the viscoelastic agent is selected from the group consisting of polyethylene glycol 400 monolaurate, polyethylene glycol 600 monolaurate, polyethylene glycol 1000 monolaurate, polyethylene glycol 4000 monolaurate, polyethylene glycol 600 dilaurate, polyethylene glycol 600 lauryl ether, and combinations thereof.

19. The formulation of claim 17 wherein the formulation comprises the viscoelastic agent in an amount of from about 0.5% (by total weight of the formulation) to about 15% (by total weight of the formulation).

20. A wet wipe for promoting skin cleanliness and health, the wet wipe comprising:
a wipe substrate; and
a formulation, the formulation comprising from about 0.01% (by total weight of the formulation) to about 10% (by total weight of the formulation) of a coefficient of friction modulator; from about 1% (by total weight of the formulation) to about 15% (by total weight of the formulation) of a moisturizer; from about 0.01% (by total weight of the formulation) to about 10% (by total weight of the formulation) of an elasticity modulator; from about 0.01% (by total weight of the formulation) to about 10% (by total weight of the formulation) of a skin texture modulator; and from about 1% (by total weight of the formulation) to about 99% (by total weight of the formulation) of a pharmaceutically acceptable carrier.

21. The wet wipe of claim 20 wherein the formulation further comprises from about 0.01% (by total weight of the formulation) to about 5% (by total weight of the formulation) of an anti-adherence agent, from about 2% (by total weight of the formulation) to about 15% (by total weight of the formulation) of a temperature modulator, and from about 0.5 (by total weight of the formulation) to about 15 (by total weight of the formulation) of a viscoelastic agent.

22. An absorbent article comprising:
an absorbent substrate; and
a formulation comprising from about 0.01% (by total weight of the formulation) to about 10% (by total weight of the formulation) of a coefficient of friction modulator; from about 1% (by total weight of the formulation) to about 15% (by total weight of the formulation) of a moisturizer; from about 0.01% (by total weight of the formulation) to about 10% (by total weight of the formulation) of an elasticity modulator; from about 0.01% (by total weight of the formulation) to about 10% (by total weight of the formulation) of a skin texture modulator; and from about 1% (by total weight of the formulation) to about 99% (by total weight of the formulation) of a pharmaceutically acceptable carrier.

23. The absorbent article of claim 22, wherein the formulation further comprises from about 0.01% (by total weight of the formulation) to about 5% (by total weight of the formulation) of an anti-adherence agent, from about 2% (by total weight of the formulation) to about 15% (by total weight of the formulation) of a temperature modulator, and from about 0.5 (by total weight of the formulation) to about 15% (by total weight of the formulation) of a viscoelastic agent.

24. The absorbent article of claim 22 wherein the absorbent article is selected from the group consisting of diapers, training pants, adult incontinence garments, feminine napkins, bath tissue, and combinations thereof.

25. A system for promoting skin cleanliness and health, the system comprising:
a formulation for promoting skin cleanliness and health, the formulation comprising from about 0.01% (by total weight of the formulation) to about 10% (by total weight of the formulation) of a coefficient of friction modulator; from about 1% (by total weight of the formulation) to about 15% (by total weight of the formulation) of a moisturizer; from about 0.01% (by total weight of the formulation) to about 10% (by total weight of the formulation) of an elasticity modulator; from about 0.01% (by total weight of the formulation) to about 10% (by total weight of the formulation) of a skin texture modulator; and from about 1% (by total weight of the formulation) to about 99% (by total weight of the formulation) of a pharmaceutically acceptable carrier;
a wet wipe comprising a wipe substrate and the formulation; or
the formulation and the wet wipe.

26. The system of claim 25 further comprising an absorbent article comprising an absorbent substrate and the formulation.

27. The system of claim 25 further comprising an absorbent article comprising an absorbent substrate and from about 0.5 (by total weight of the formulation) to about 15% (by total weight of the formulation) of a viscoelastic agent, from about 0.01% (by total weight of the formulation) to about 5% (by total weight of the formulation) of an anti-adherence agent, or combinations thereof.
